(19) United States
(12) Patent Application Publication
(10) Pub. No.: US 2019/0038226 A1
(43) Pub. Date: Feb. 7, 2019

(54) POLYMER GEL PRODUCTS WITH PHYSIOLOGICAL AND BIOMECHANICAL BENEFITS AND/OR MONITORING

(71) Applicant: Hexyoo Scientific Inc., Toronto (CA)

(72) Inventors: Peter L. Davidson, Aurora (CA); Robert L. MacMillan, Courtice (CA); Kristopher James Edwards, Oshawa (CA); Michael W.R. Holmes, Fonthill (CA); Jason R. Thomson, Aurora (CA); Ross Libbey, Courtice (CA)

(21) Appl. No.: 16/070,538

(22) PCT Filed: Jan. 17, 2017
(86) PCT No.: PCT/CA2017/050051

§ 371 Date: Jul. 17, 2018

Related U.S. Application Data
(60) Provisional application No. 62/279,795, filed on Jan. 17, 2016.

(51) Int. Cl. A61B 5/00; A61F 7/02; A61F 13/00; A61F 13/06; A61F 5/34; B29C 45/14; B29C 59/02; B29C 45/26

(52) U.S. Cl. CPC A61B 5/6831; A61B 5/6843; A61B 5/447; A61F 7/02; A61F 13/00017; A61F 2007/0096; A61F 5/34; B29C 45/14008; B29C 59/026; B29C 45/263; A61F 2007/0219; A61F 13/069

(57) ABSTRACT

Various embodiments of polymer gel products are described herein. A support pad comprising a viscoelastic polymer based gel substrate having a selected thickness and flexibility, and at least one sensor embedded within the gel substrate proximal to the surface of the support pad that is adjacent to the portion of the user's body. A method of embedding at least one sensor into a vis-polymer gel substrate. An impact dissipating pad insertable into a body protection apparatus and comprising a viscoelastic polymer based gel substrate having a selected thickness and first and second surfaces. A compression wrap comprising a gel band that is stretchable and a backing member adjacent to the gel band, wherein the backing member is stretchable in a similar manner as the gel band. An anti-vibration glove comprising at least one gel material disposed in the glove to cover at least a portion of the user's hand.

### CROSS-REFERENCE TO RELATED APPLICATION

This application claims the benefit of U.S. Provisional Patent Application No. 62/279,795 filed Jan. 17, 2016; the entire contents of Patent Application No. 62/279,795 are hereby incorporated by reference.

### FIELD

Various embodiments are described herein for polymer gel products for various uses including at least one of health and wellness monitoring and therapeutic applications.

### BACKGROUND

Health and wellness has always been an important issue and is only gaining more importance with the increasing aging population and with today's sedentary lifestyle. Various products have been developed to improve health and wellness but typically use conventional materials that result in products that are cumbersome or uncomfortable to use and do not have a high level of effectiveness.

### SUMMARY OF VARIOUS EMBODIMENTS

In another broad aspect, at least one embodiment described herein provides a support pad comprising: a viscoelastic polymer based gel substrate having a selected thickness and first and second opposing surfaces, and wherein the gel substrate has a flexibility to conform to a portion of a user's body that is in contact with the support pad during use; and at least one sensor embedded within the gel substrate proximal to the surface of the support pad that is adjacent to the portion of the user's body during use.

In at least one embodiment, the viscoelastic polymer gel substrate further comprises one or more additives for modifying one or more physical characteristics of the gel substrate.

In at least one embodiment, at least one of the first and second surfaces are textured.

In at least one embodiment, the textured surface comprises at least one chevron pattern, at least one linear channel pattern, a pattern of circular ridges, a pattern of solid circular ridges or a combination pattern having chevrons and linear channels.

In at least one embodiment, a plurality of air pockets are disposed in the textured surface.

In at least one embodiment, the textured surface comprises at least one drainage port to drain liquids during use.

In at least one embodiment, the at least one drainage port is coupled to at least one channel in the textured surface.

In at least one embodiment, the at least one sensor comprises at least one of a pressure sensor, a force sensor, a temperature sensor, an accelerometer and a light sensor.

In at least one embodiment, the at least one sensor is embedded in a gel substrate that is proximal to a user's body surface during use.

In at least one embodiment, the support pad further comprises an interface module coupled to the at least one sensor, and during use, the at least one sensor senses information about the user and/or support pad and the interface module communicates the sensed information to a receiver module via wired or wireless communication.

In at least one embodiment, the support pad further comprises one or more of at least one light emitting diode that is embedded in the gel substrate to promote localized tissue healing during use and at least one ultrasound transducer that is embedded in the gel substrate to provide thermal and/or vibration therapy during use.

In at least one embodiment, the support pad is a mattress topper or a mattress insert and the gel substrate is adapted to conform to the shape of the region of the user's body resting on the support pad during use.

In at least one embodiment, the support pad is used, on a bed and the at least one sensor comprises an array of pressure sensors embedded in the gel substrate and the sensors comprise a greater sensitivity when located at one or more areas that correspond with one or more of the user's heels, hips, elbows, shoulders and back of their head.

In at least one embodiment, the at least one sensor of the support pad is in communication with a sleep monitoring system configured to identify at least one sleep disorder for the user and the gel substrate is adapted to reduce at least one sleep-disrupting factor associated with a conventional sleep support.

In at least one embodiment, the support pad is a motor vehicle seat or back insert or a motor vehicle seat support pad and the gel substrate is adapted to dissipate low level whole body vibration during use.

In at least one embodiment, a foam layer is adjacent to at least one of the first and second surfaces of the gel substrate.

In at least one embodiment, wires used with the at least one sensor are located in a channel formed within the gel substrate.

In at least one embodiment, wires used with the at least one sensor is located along one section of the support pad where the least amount of bending and twisting occurs during use.

In at least one embodiment, anchors are coupled to wires used with the at least one sensor to ease stress on the wires and the connections between the wires and the at least one sensor.

In at least one embodiment, the at least one sensor is held in place by a support material that is flexible and does not affect the performance of the at least one sensor.

In another broad aspect, at least one embodiment described herein provides an impact dissipating pad to provide protection to a user's body part during use, the impact dissipating pad comprising a viscoelastic polymer based gel substrate having a selected thickness and first and second surfaces, wherein the impact dissipating pad is insertable into a body protection apparatus.

In at least one embodiment, the impact dissipating pad further comprises at least one sensor embedded within the gel substrate proximal to the surface of the gel substrate that is closest to the user's body.

In at least one embodiment, the at least one sensor comprises at least one of a pressure sensor, a force sensor, a temperature sensor, an accelerometer and a light sensor.

In at least one embodiment, the at least one sensor comprises at least one of a pressure sensor and an accelerometer embedded proximal to the surface of the impact dissipating pad to facilitate measurement of a magnitude of an impact when the impact dissipating pad experiences the impact during use.

In at least one embodiment, the impact dissipating pad further comprises an interface module coupled to the at least one sensor, and during use, the at least one sensor senses information and the interface module communicates the sensed information to a receiver module via wired or wireless communication.

In another broad aspect, at least one embodiment described herein provides a method of embedding at least one sensor into a viscoelastic polymer gel substrate, the method comprises: placing a support structure for the at least one sensor into a mold; placing the at least one sensor on the support structure; heating and processing a slurry comprising a mixture of polymer and oil to a desired temperature to produce a viscoelastic gel liquid; inserting the gel liquid into the mold; and allowing the gel liquid to set to form a solid viscoelastic polymer gel with the at least one embedded sensor embedded therein. This process may be thermo-reversible in nature.

In at least one embodiment, the method further comprises adding one or more additives to the slurry for modifying one or more physical characteristics of the solid viscoelastic polymer gel.

In at least one embodiment, the method further comprises adding additional electronics to the at least one sensor in the mold and establishing electrical connections prior to dispensing the liquid gel material into the mold.

In at least one embodiment, the method further comprises texturing a receiving surface of the mold to produce a textured surface on the solid viscoelastic polymer gel.

In at least one embodiment, the texturing comprises forming at least one chevron pattern, at least one linear channel pattern, a pattern of circular ridges, a pattern of solid circular ridges or a combination pattern having chevrons and linear channels on the textured surface.

In at least one embodiment, the method comprises forming a plurality of air pockets on the textured surface.

In at least one embodiment, after the liquid gel has been poured into the mold up to a selected thickness and the liquid gel is nearly set the support structure is removed.

In at least one embodiment, the support structure is made from steel or aluminum formed as a pin such that it supports but is not permanently attached to the at least one sensor.

In at least one embodiment, prior to placing the support structure into the mold a first layer of gel liquid is poured, the at least one sensor is attached to a flexible piece of material and placed on the first layer of gel when the first layer of gel sets or nearly sets, and a new layer of gel is poured over the flexible piece of material such that the new layer of gel has a selected thickness and covers the at least one sensor.

In at least one embodiment, the flexible piece of material comprises fabric or another flexible material.

In at least one embodiment, after the gel is set, the method comprises injecting a foam mixture which rises and expands to fill the mold to form a gel injected foam product.

In at least one embodiment, the method comprises using a contoured mold to provide a contour to the gel and foam portions of the gel injected foam product.

In a broad aspect, at least one embodiment described herein provides a compression wrap comprising: a gel band that is stretchable and has first and second surfaces and first and second end portions; and a backing member adjacent to one of the first and second surfaces of the gel band, wherein the backing member is stretchable in a similar manner as the gel band.

In at least one embodiment, the gel band comprises viscoelastic polymers.

In at least one embodiment, the gel band further comprises one or more additives for modifying one or more physical characteristics of the gel band.

In at least one embodiment, the backing member is permanently attached to the gel band.

In at least one embodiment, the backing member is removable from the gel band and is re-attachable after use for storage.

In at least one embodiment, the gel band is self-adhering.

In at least one embodiment, the backing member comprises at least one of a fabric material including one or more of polyester, rayon, spandex, and nylon.

In at least one embodiment, the gel band has an elasticity to enable deformation in the range of 2:1 to 10:1 to provide a sufficient compressive force when the compression wrap is wrapped around an anatomical location of a user.

In at least one embodiment, the gel band has a thermal conductivity in the range of 4.0 to 6.0 Watts per meter Kelvin (W/(m·K)) to allow the gel band to maintain a desired temperature over a desired period of time when the compression wrap is chilled or heated prior to being wrapped around the anatomical location of the user.

In at least one embodiment, the compression wrap further comprises at least one sensor embedded within the gel band.

In at least one embodiment, the at least one sensor comprises at least one of a pressure sensor, a force sensor, a temperature sensor, an accelerometer and a light sensor.

In at least one embodiment, the at least one sensor is embedded proximally to the second surface of the gel band, the second surface being placed adjacent to a surface of a user during use to facilitate measurements from the user.

In at least one embodiment, the at least one sensor comprises an interface module, and during use, the at least one sensor senses information and the interface module communicates the sensed information to a receiver module via wired or wireless communication.

In at least one embodiment, the sensors are embedded near a first end of the band, wherein the first end is applied to the body part first.

In another broad aspect, at least one embodiment described herein provides a compression wrap comprising: a gel band that is stretchable and has first and second surfaces and first and second end portions, wherein the gel band comprises a viscoelastic polymer that adheres to itself allowing the compression wrap to be wrapped around a body part and maintained in position while providing compression due to the self-adhesion of the gel band.

In another broad aspect, at least one example embodiment is provided for an anti-vibration glove for reducing vibrations experienced by a user of the anti-vibration glove, wherein the anti-vibration glove comprises at least one gel material disposed in the glove to cover at least a portion of the user's hand and reduce vibrations experienced by the user at the portion during use.

In at least one embodiment, the glove comprises a gel substrate that covers a substantial portion of the glove that is in contact with the inner surface of the user's hand.

In at least one embodiment, the glove comprises a gel pad that is disposed along a cuff portion of the glove that is in contact with the inner surface of the user's wrist during use.

In at least one embodiment, the glove comprises at least one gel pad that is disposed on a lower palm portion of the glove.

In at least one embodiment, the at least one gel pad is disposed at the thumb regions of the glove.

In at least one embodiment, the glove comprises a single gel pad that is disposed on an upper palm portion of the glove and has extension regions that extend along a portion of the proximal finger portions of the glove.

In at least one embodiment, the glove comprises several gel pads that are disposed along an upper palm portion of the glove and are separate from one another.

In at least one embodiment, the glove comprises several gel pads disposed along a distal portion, medial portion and proximal portion of at least one finger of the glove.

In at least one embodiment, the glove comprises several gel pads disposed along a distal portion, medial portion and proximal portion of each finger of the glove.

In at least one embodiment, the gel pads are disposed along the finger regions of the glove are separate or connected to one another by additional gel pads.

In at least one embodiment, the gel substrate comprises additional gel pads to provide additional vibrational dampening, the additional gel pads being disposed at one or more of the lower palm, the upper palm and the finger regions of the glove substrate.

In at least one embodiment, the additional gel pads are formed integrally with the gel substrate or additional gel layers formed on the gel substrate.

In at least one embodiment, the additional gel pads are separate pieces of gel material made formed on the gel substrate, the gel substrate being made of one gel composition and at least one of the additional gel pads being made of a second different gel composition based on the location of the at least one additional gel pad and/or the application of the glove.

In at least one embodiment, the at least one gel material has a textured surface to provide further vibrational dampening.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now briefly described. The drawings are not intended to limit the scope of the teachings described herein.

FIGS. 1A-1C show a top view, a side view and a perspective view of an example embodiment of a compression wrap made of a polymer gel band in accordance with the teachings herein.

FIG. 2A shows a bottom view and a top view of an example embodiment of a compression wrap having a polymer gel band with a smooth surface and a backing member in accordance with the teachings herein.

FIG. 2B shows a bottom view and a top view of an example embodiment of a compression wrap having a polymer gel band with a textured surface and a backing member in accordance with the teachings herein.

FIG. 2C shows a side view of an example embodiment of a compression wrap having a polymer gel band and a backing member in accordance with the teachings herein in which an end of the compression wrap is rolled out and oriented vertically.

FIGS. 3A-3C show a top view, a side view and a perspective view of an example embodiment of a compression wrap with embedded sensors in accordance with the teachings herein.

FIGS. 4A-4B show a top view and a perspective view (or cross-sectional perspective view), respectively, of a polymer gel support pad with embedded sensors in accordance with the teachings herein.

FIG. 5 is a block diagram of an example embodiment of a processing line that may be used to produce polymer gel and polymer gel products in accordance with the teachings herein.

FIG. 6 is a flow chart of an example embodiment of a method for producing polymer gel and polymer gel products in accordance with the teachings herein.

FIGS. 7A-7D are images of example embodiments of anti-vibration gloves (or vibration reducing gloves) or parts of these gloves with gel pads for reducing or absorbing vibrations in accordance with the teachings herein.

FIG. 8A is an image of an example embodiment of a support pad with a surface that has a pattern of chevrons and linear channels.

FIGS. 8B-8C show perspective views of two example embodiments of a support pad with a surface that has a pattern of circular ridges.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to products or methods having all of the features of any product or method described below or to features common to several or all of the products and methods described herein. It is possible that there may be a product or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that, as used herein, the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof that are modified by the term “about” is presumed to means that a variation is possible of up to a certain amount of the number to which reference is being made if the end result is not significantly changed, such as 10%, for example.

The teachings herein relate to polymer-based slurries that may be processed using innovative processing techniques to obtain a variety of polymer gel components having desired properties such as, but not limited to, a certain amount of firmness and/or elasticity, for example, that make the polymer gel products suitable for at least one of health, wellness, therapeutic and/or monitoring applications. For example, the elasticity of the gel material may be selected to allow the polymer gel product to return to its original shape after being compressed, stretched or otherwise physically manipulated. These polymer gel products include, but are not limited to, support pads for the prevention of pressure ulcers and stretchable compression wraps for compression and/or thermal therapy of physical injuries.

The polymer gel products, in which the polymer gel forms a substantial portion of the product and which described in accordance with the teachings herein, may be manufactured to attain various degrees of firmness, elasticity, elongation, tensile strength, tear resistance and adhesive properties as well as other physical or thermal characteristics by starting with a certain slurry formulation including polymers and adjusting various parameters of a gel-producing process to obtain a soft ViscoElastic Polymer (VEP)—based solid product. In some embodiments, the upper surface of the solid product may comprise one or more channels or grooves to provide certain properties such as increased flexibility and/or pressure dissipation. Differing material properties (e.g., creep, firmness, stiffness, and elasticity) of the resultant gels may result in one or more benefits such as, but not limited to, one or more of pressure dissipation, body temperature regulation, body support and localized application of compressive forces, for example. In some embodiments, gel materials comprising VEPs may be used in accordance with the teachings herein, and may be preferable due to their inherent lateral elasticity and thermal conductivity (allowing for improved heat dissipation).

A gel material comprising VEPs may be considered a viscoelastic substance having elastic and viscous attributes when undergoing deformation. The elastic attribute permits the gel to be stretched out (i.e. imparting a strain on the gel material) and return to its original state when it is no longer stretched. The viscous attributes may be selected to allow the gel material to dissipate energy when a load is applied thereby giving the gel shock absorbing and vibration reducing qualities. According to the teachings herein, the use of viscoelastic gel material may be beneficial in a number of health, wellness, travel and sleep products and applications.

Pressure ulcers or bedsores are common amongst those who experience limited mobility for an extended period of time. Such ulcers are a localized injury to a portion of the surface of the skin or underlying tissue due to continuous pressure and shear forces that are acting at the ulcer location due to contact with a surface. Individuals who sit for an extended period of time (e.g. truck drivers, long distance bus drivers), those that are confined to a wheelchair, or those who are bedridden may be particularly susceptible to pressure ulcers. Bedridden individuals may suffer from pressure ulcers due to the pressure and shear forces imparted on the body at the body-mattress interface. Pressure ulcers are recognized as a serious health care problem that increases healthcare costs as a result of additional treatment that is required. For example, pressure ulcers can increase the length of a hospital stay which further increases the cost of providing healthcare to affected individuals. Therefore, greater attention to the prevention of pressure ulcers is needed, warranting the creation of products capable of alleviating the pressure induced on the body by a support surface such as a mattress for example that may lead to the development of pressure ulcers.

In some instances, however, localized pressure may be desirable for treating a body part that has experienced physical trauma. For example, it is generally desirable to apply constant and localized pressure to strained or sprained muscles to control blood flow and restrict further muscle swelling. Other examples in which the application of pressure may be used include splinting or immobilization of an injured body part to prevent further injury. In some situations, it may also be necessary to alter the applied pressure over time, such that a mechanism for detecting the applied pressure is desirable. In other instances, it may also be desirable to apply heat or cold therapy in addition to the applied pressure to promote healing of the injured body part.

In these various applications, VEP gel products in which the VEP gel is the substrate or VEP gel components in which the VEP gel is a component of the final product, produced in accordance with the teachings herein, may be used as the primary material in the fabrication of support and compression products for various health, wellness and therapeutic applications.

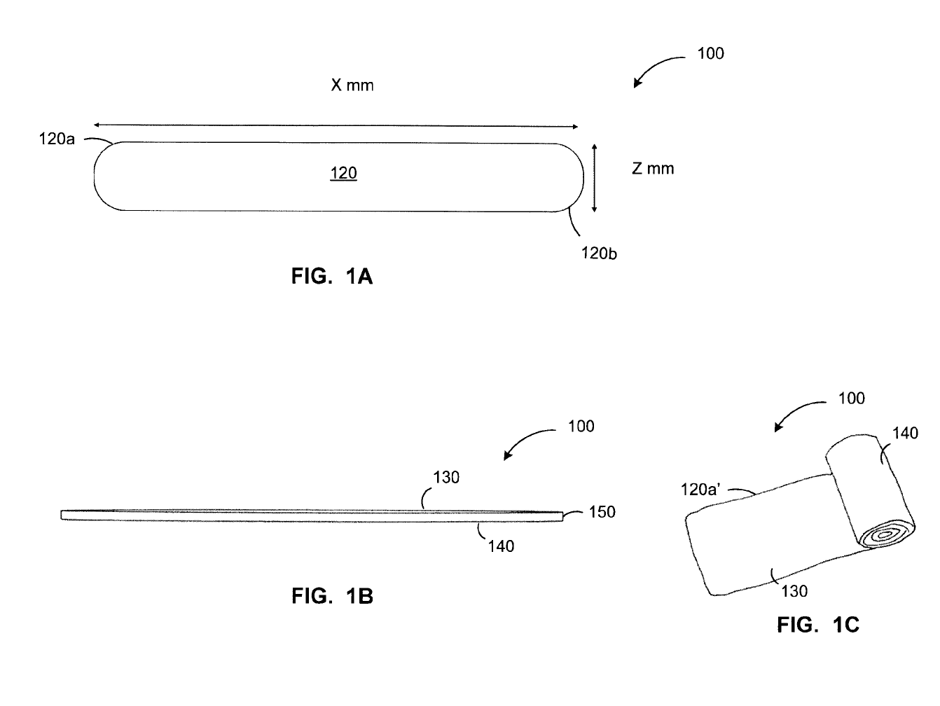

Referring now to FIGS. 1A-1C, shown therein are various views of an example embodiment of a compression wrap 100 consisting of a gel band 120 in accordance with the teachings herein. The gel band 120 has a first surface 130 and a second surface 140 that is opposite of the first surface 130. The compression wrap 100 may be used as a compression bandage that may be physically wrapped around an anatomical location of a user. The anatomical location may be a body part such as a joint, a boney structure or a muscle. For example, the body part may be a wrist, a forearm, a bicep, an elbow, a thigh, a calf, and a foot. The user may be a regular person or animal or a person or animal that is an athlete.

A polymer may be formulated to produce the gel band 120 such that the compression wrap 100 has a selected degree of elasticity so as to permit the compression wrap 100 to stretch beyond its resting length (i.e. the length of the compression wrap 100 when it is in a non-stretched or relaxed state). The degree of stretching may be quantified based on the ratio between the achievable length (e.g. “stretched” position) of the gel band 120 when stretched and the resting length (e.g. “resting” position). For example in some embodiments, the gel band 120 may have a stretch ratio of 2:1. In other embodiments, the gel band may have a stretch ratio that increases from 2:1 to 10:1. The gel material can be formulated and manufactured to have a certain modulus of elasticity to provide a selected degree of elasticity. The formulation and manufacture of the gel material can be varied to permit the amount of compression to be easily varied.

In at least some embodiments, a polymer formulation may be used to create a VEP gel that combines the benefits of a selected rate of temperature change (e.g. hot/cold) with a selected amount of compression for therapeutic benefit, in accordance with the teachings herein. This is in contrast with conventional wraps that may require additional components for cooling, such as the the addition of water, or may require additional components for compression, such as a Velcro™ strap, or may provide compression without the benefit of cooling or heating.

In some cases, cryotherapy (i.e. treating with ice or cooling) may be preferred for the treatment of acute injuries to decrease the internal temperature of the injured tissue (e.g. muscle) to slow down the tissue's metabolic rate. Compression may generally be introduced in conjunction with cryotherapy for injury management to reduce the chance of developing edema of the injured tissue. The steady and continuous compression combined with temperature variation may improve clinical outcomes for both professional and home uses. In particular, it is generally understood that compression greater than 30 to 40 mmHg may reduce blood flow (Ashton H., “The effect of increased tissue pressure on blood flow”, Clin Orthop., 1975, 113: 15-26) to generate a therapeutic effect; however exact pressure numbers are challenging to confirm, since this can be body part specific, individualized and depends on methods of pressure measurement. Often, lower pressure can be desired in other body areas or body parts. The gel band 120 can be made to achieve such compression. In other embodiments, the gel band 120 can be made to achieve a larger range of compression. The elasticity of the gel band 120 allows for the compression wrap 100 to be applied to any anatomical location for direct application with full coverage, comfort, and compression at the desired pressure range.

The gel band 120 may be molded or shaped to have a selected thickness 150 (as well as a certain resting length and resting width) to prevent the gel band 120 from tearing while it is stretched. Dimensions for the gel band 120 may range from one-eighth of an inch to one inch or more (if desired) in thickness, from one inch to 12 inches or more (if desired) in width and up to 12 inches or more (if desired) in length. For example, the gel band 120 may be made in different sizes to accommodate users of different size. For example, the gel band 120 may come in small, medium and large sizes. All of these sizes may have the same thickness, for example about ¼ inch, lengths of about 30, 48 or 72 inches and widths of about 3, 4.5 and 6 inches, for the small, medium and large sizes, respectively or any reasonable combination thereof.

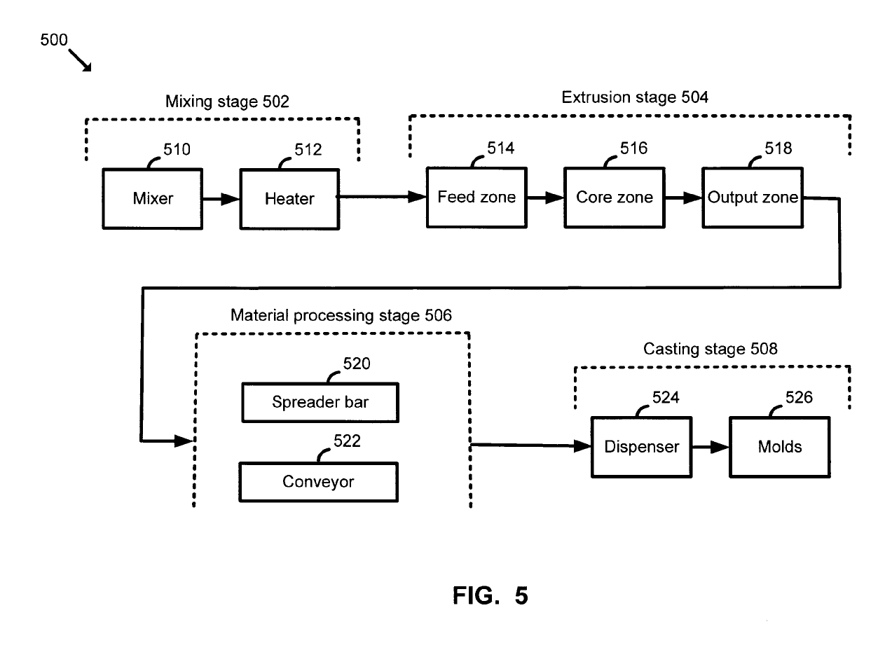

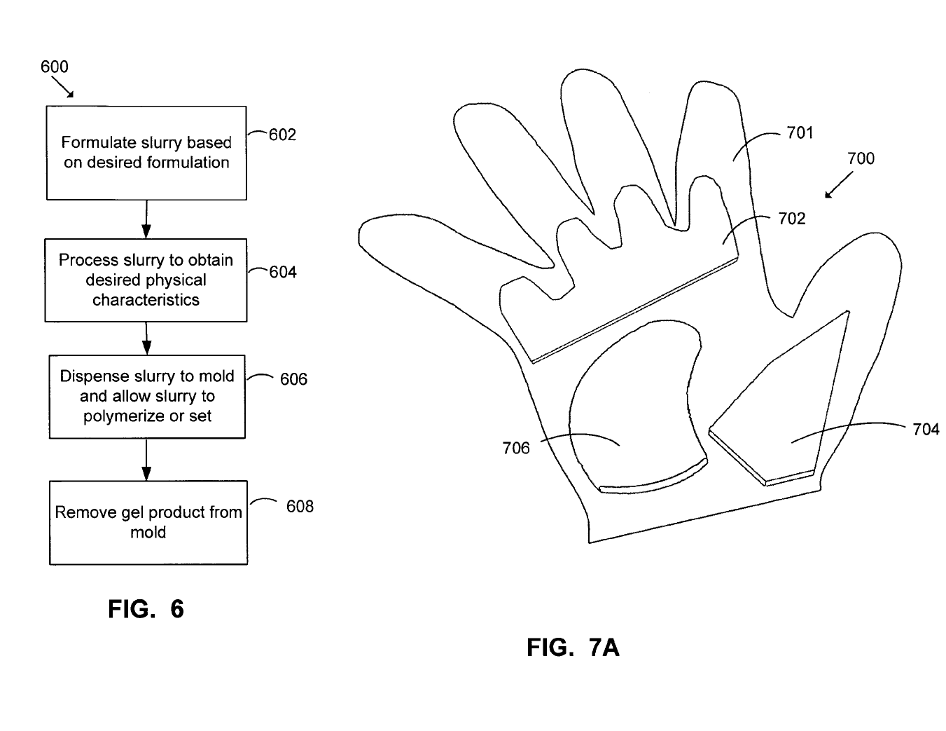

The elastic nature of the compression wrap 100 provides flexibility to permit physical manipulation of the compression wrap 100 so that it may be wrapped around any exterior anatomical location to provide compressive pressure thereto. For example, the compression wrap 100 may be wrapped around the wrist, arm or leg or any desired external body part to provide steady and continuous compressive pressure to the area surrounded by the compression wrap 100. The degree of applied pressure or compression may be adjusted by changing the extent of which the gel band 120 is stretched and wrapped around the body part and also depending on the formulation and processing of the VEP gel material described in further detail below with respect to FIGS. 5 and 6.

In some embodiments it is also desired that the compression wrap 100 provides thermal therapy when it is wrapped around a body part. Accordingly, the gel band 120 may be formulated from a certain polymer formulation in such a way that it has a selected thermal conductivity which allows it to be cooled (e.g. placed in a refrigerator, freezer or in any environment in which the temperature is lower than that of the VEP gel material) or heated (e.g. placed in a hot water bath, water bath in a heated environment where the temperature is greater than that of the VEP gel material and heat can be transferred to the VEP gel material) so that the compression wrap 100 provides a desired amount of heat or cold for a certain amount of time to the body part that is surrounded by the compression wrap 100 during use. The VEP gel material may be produced so that the compression wrap 100 has a selected coefficient of thermal conductivity that allows the compression wrap 100 to become warm over a certain period of time thus providing treatment while avoiding other problems like frostbite (if the compression wrap 100 is initially cooled prior to use). For example, the thermal conductivity may be selected to be in the range of about 4.0 to 6.0 Watts per meter Kelvin (W/(m·K)). In other instances, it may be preferable to produce a VEP gel material such that it remains pliable when cooled so that the cooled compression wrap 100 may be applied to the target body part. In some embodiments, the formulation selected to manufacture the VEP gel material may remain pliable down to −20° C. (−4° F.) since the components used to make the VEP gel material have freezing points lower than −20° C. (−4° F.). In other embodiments, the components used to make the VEP gel material can be selected to have freezing points lower than −5° C. (23° F.) to −20° C. (−4° F.). This is beneficial as it has been found that a temperature reduction of 10 to 15° C. (a change of 18 to 27° F.) may provide therapeutic benefit (MacAuley, D., “Ice therapy: How good is the evidence?”, Int. J. Sports Med., 2001, 22(5): 379-384).

In at least some embodiments, the gel band 120 may be made from a polymer formulation such that it is self-adhering so that when the compression wrap 100 is wrapped around an external body part, the surface portions of the gel band 120 that are adjacent to one another may adhere or grip to one another wrap so that the compression wrap 100 stays in place while providing a desired amount of compression without using any additional binding mechanisms such as a safety pin or straps, for example. This self-adhesion property allows for better support and more uniform cooling of the body part that is being treated. Additionally, the pressure displacing properties of the selected VEP gel material may further provide force dampening properties to reduce damage due to impact forces to the injured body part when the compression wrap 100 is being worn.

In at least some embodiments, the compression wrap 100 may be made such that it can be rolled into a compact form while it is not in use (e.g. during storage, while being chilled or heated), as shown in FIG. 1C, so as to reduce the physical footprint of the compression wrap 100 and then unrolled to be applied to a body part. In these cases, the polymer material formulation and the manufacturing process to produce the VEP gel band may be adjusted so that the adhesive properties of the gel band 120 allows it to be easily rolled and unrolled.

In at least some embodiments, the VEP gel material may be produced using polymer formulations that allow the material to be cut during manufacture to produce custom, body part specific designs that are pre-manufactured based on the anthropometry and anatomy of the user. This VEP gel material design allows that the compression wrap 100 to be form fitting to the user for more effective treatment. The VEP gel material can be cut without fear of the material losing its inherent shape or leaking of any fluids since the gel material is a solid material—it is not a liquid and is not held in a bladder. Therefore, even the end user may trim the final VEL gel product without fear of an unknown fluid leaking from the cut point.

In at least some embodiments, an example of which is shown in FIG. 1A, at least one of the end portions 120a and 120b of the gel band 120 may be tapered to assist with manipulation of the gel band 120. Alternatively at least one of the end portions 120a and 120b for compression wrap 100 may have a non-tapered rectangular shape. Tapering the end portions of the compression wrap 100 may provide ergonomic advantages. For example, a compression wrap 100 with a tapered end may allow for an easy starting point for wrapping certain anatomical areas so that the therapeutic application of the compression wrap 100 may begin at a localized point and the wrapping may progress outward, around the injury to cover a larger anatomical area. The overall effect of this shape may result in enhanced user comfort and ease of application.

In at least some embodiments, the gel band 120 of the compression wrap 100 may be textured on the first surface 130 and non-textured on the second surface 140 or vice-versa, or both the first and second surfaces 130 and 140 may be textured. The texturizing may be done using various techniques and various types of textures as described herein. Texturizing at least one surface of the gel band 120 may enhance user comfort by improving conformance to the user's body part, making the gel band 120 easier to apply or wrap around a body part and increasing breathability of the gel material. Furthermore, texturized surfaces may facilitate dissipation of excess heat and better distribution of weight/pressure as compared to comparable gel materials that lack texture. This applies not only to the compression wrap embodiments, but to the support pad, insert pad, impact pad and other gel products described in accordance with the teachings herein.

In some cases, when the gel band 120 is rolled up it may be difficult to unroll if the gel band 100 has a certain amount of surface adhesion. Furthermore, in some cases when the gel band 120 is rolled up and placed in a freezer for cooling, the adjacent layers of the gel band 120 that are rolled together may freeze making the unrolling process more difficult. In accordance with the teachings herein, in at least some embodiments of the compression wrap there may be a second additional layer such as a backing layer or backing member.

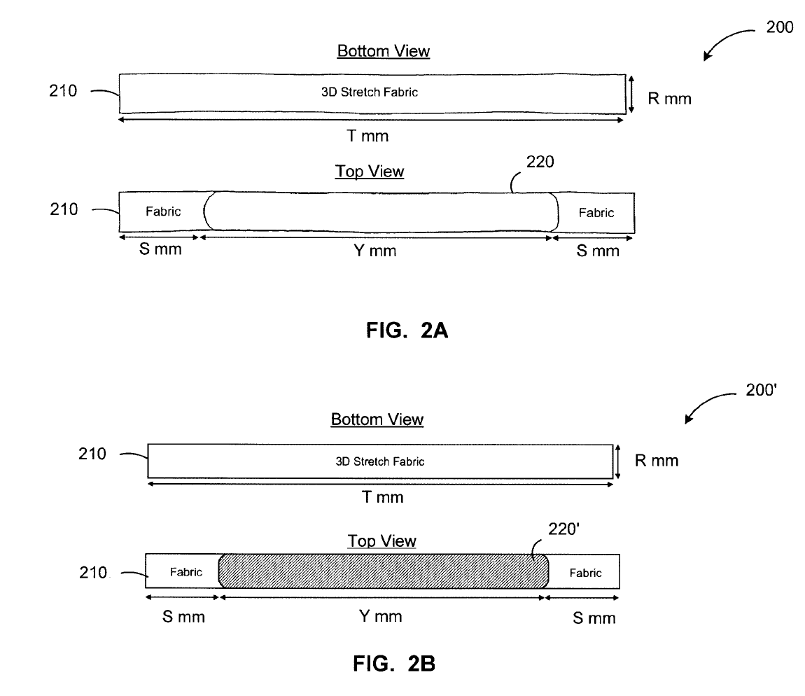

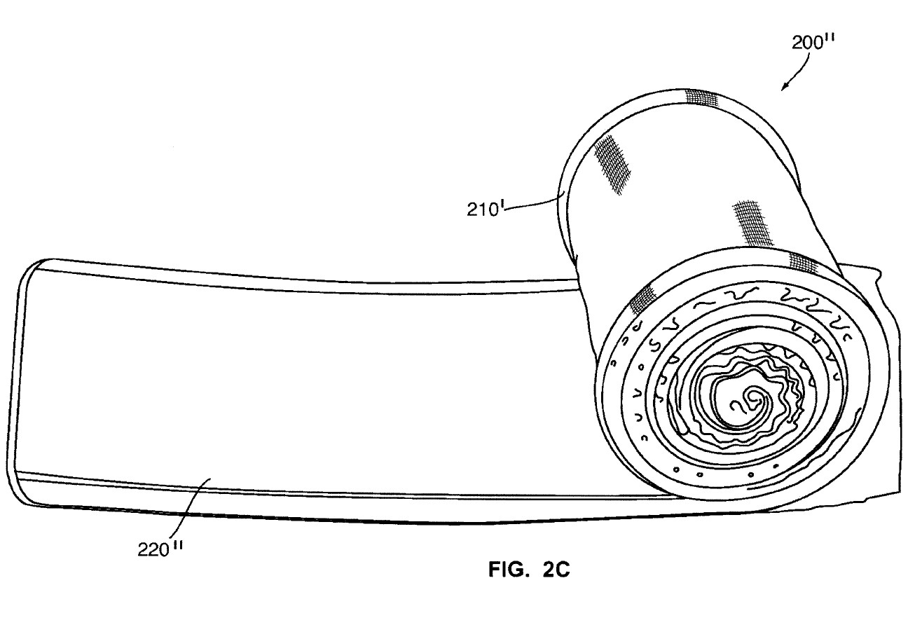

Referring now to FIGS. 2A-2C, shown therein are various views of an example embodiment of a compression wrap 200, 200′ having a gel band 220, 220′ and a backing member 210 in accordance with the teachings herein. The gel band 220, 220′ is self-adhering and the backing member 210 is used to facilitate the rolling and unrolling of the compression wrap 200. The backing member 210 provides a support layer upon which the gel band 220 or the gel band 220′ is placed. The surface of the gel band 220 that is opposite the backing member is soft while the surface of the gel band 220′ that is opposite the backing member 210 is textured. As another example, FIG. 2C shows an embodiment of a compression wrap 200″ having a gel band 220″ with a textured surface which is rolled in a jelly wrap configuration such that the gel band 220″ comes into contact with the backing member 210′ so that it does not adhere to itself when it is rolled up.

The backing member 210 may be made from material that is flexible so that it can be rolled. In some cases, the backing member 210 may be made from a stretchable material to permit manipulation of the compression wrap 200. The backing member 210 may also be made with a material that may be used to prevent adjacent layers of the self-adhering gel material 220, 220′ or 220″ from sticking to each other or freezing to one another during use when in a rolled configuration. In some example embodiments, the backing member 210 may be a stretch fabric made of one or more of polyester, Rayon, Spandex, Nylon and Spandex permitting movement and stretching in any direction. For example, the backing member 210 may be made of Ponte di Roma, heavy, 72% Polyester, 23% Rayon, and 5% Spandex, or 80% Nylon and 20% Spandex comprising 80% nylon and 20% Spandex. In other example embodiments, the backing material may be chosen for its thermal properties. For example, the desired backing material may be chosen to be a thermal insulator so that when the compression wrap 200 is heated or cooled, the backing material may further reduce the rate of heating or cooling when the gel wrap is not in use. The backing material may also be chemically treated so that the backing is anti-bacterial or anti-microbial. In some embodiments, the backing material may also be selected to limit the amount of compression as the capability of the gel band to compress is limited by the stretchability of the backing material when the backing material is attached to the gel band.

In some embodiments, the gel band 220, 220′ or 220″ may be permanently bonded to the backing member 210. For example, polymers can be applied to the backing member 210 prior to polymerization so as to allow some of the polymers to impregnate the backing member 210 and create a permanent bond with the gel band 220, 220′ or 220″ during production. In this case, one surface of the gel band 220, 220′ or 220″ is permanently bonded to the backing member 210 while the other surface of the gel band 220, 220′ or 220″ can be applied adjacent to a user's external body part so as to apply thermal therapy as explained previously for the compression wrap 100. In these embodiments, the backing member 210 and the gel band 220, 220′ or 220″ can provide compression when the compression wrap 200 is applied to the user's external body part.

In other embodiments, the attachment of the backing member 210 to the gel band 220, 220′ or 220″ is a temporary backing such that the gel band 220, 220′ or 220′ may be separated from the backing member 210 during use such as after the compression wrap 200 is in an unrolled configuration prior to being applied to a user. In some embodiments, the backing member 210 may be configured so that the gel band 220, 220′ or 220″ may be subsequently re-attached to the backing member 210 after they have been separated from one another. To make the backing member 210 re-attachable, the backing material may be fabricated separately from the gel band 220, 220′ or 220″, then combined with the gel band 220 or 220′ and rolled up for storage or cooling/heating. The re-attachable backing material may reduce the tendency of the gel band 220, 220′ or 220″ to self-adhere, for example, when it is cooled or frozen to enhance its ability to be manipulated.

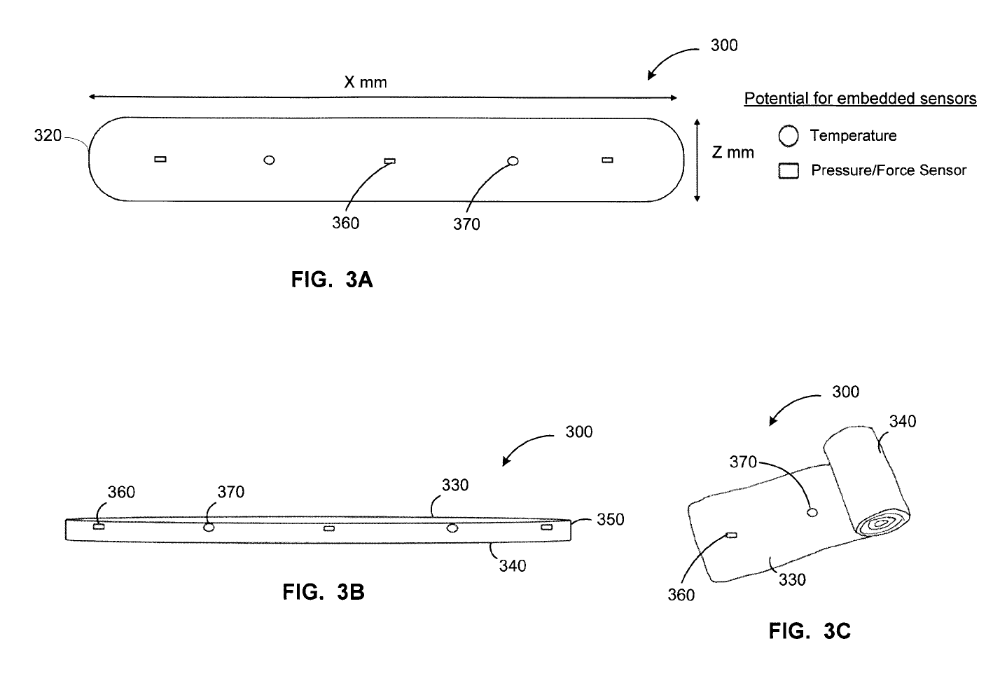

Referring now to FIGS. 3A-3C, shown therein are various views of an example embodiment of a compression wrap 300 with embedded sensors 360, 370 in accordance with the teachings herein. The embedded sensors 360, 370 may be used to monitor one or more physiological and biomechanical parameters of the user of the compression wrap 300. Various types of sensors may be used so that more than one type of data can be collected for a user simultaneously or sequentially. In this example embodiment, there are two types of sensors but more or less types of sensors may be used and there may be one or more of each type of sensor in the compression wrap. The type of sensors used and the number of each sensor type that is used depend on the type of information that will be measured from the user.

For example, in some embodiments of the compression wrap 300, the sensors may include one or more pressure/force sensors 360, one or more temperature sensors 370, and/or one or more optical elements (not shown). If at least two of a given sensor type are used, they may be distributed horizontally across the length of the wrap as shown in FIG. 3A or they may be oriented in a vertical fashion in a column (not shown) at one longitudinal location along the wrap 300. The pressure/force sensors 360 and temperature sensors 370 may be used to measure a localized compressive force that is applied by the compression wrap 300 or a localized skin temperature, respectively. The optical elements may be used for diagnostic applications. For example, optical emitters and optical sensors may be embedded within the gel material to generate a photoplethysmogram to monitor blood circulation or heart rate by detecting time-based changes in tissue optical properties due to blood flow in the portion of the body surrounded by the compression wrap 300.

In at least some embodiments, the sensor output may be transmitted to a receiver for display or further processing. The receiver may be a smartphone, or other computing device, operating a compatible application configured to communicate with the sensors and receive sensed data. For example, with respect to the force/compression sensors, the application may indicate the optimal pressure for a given therapeutic objective (e.g. 30-40 mmHg to reduce blood flow for improved healing) and display the current compressive force being applied. This feature may enable the user or healthcare provider to determine whether or not the compression wrap should be adjusted to obtain the desired amount of compression. Alternatively, if the change in time of the pressure measurements show a reduction in pressure below a first selected pressure threshold then the wrap 300 may be getting looser and needs to be tightened. In yet another alternative, if the pressure measurements is higher than a second selected pressure threshold then a warning can go off so that the compression wrap 300 can be loosened and re-applied at a lower pressure to avoid having the user develop blood clots.

If more than one sensor of the same sensor type is used, individual sensor readings may be displayed or used individually or they may be averaged and then displayed or further processed in some embodiments.

In some embodiments at least one of the embedded sensors may be used as an alert system. For example, a person wearing the compression wrap 300 who falls may trigger a sudden spike in the detected force/pressure. One or more of frequency filtering and amplitude filtering can be used to identify the sudden peak. For example, raw measured data can be band pass filtered and optionally digitally low pass filtered to remove high frequency content that is likely noise in the measured signal. Then, a baseline reading can be used to set a user specific threshold (e.g. the threshold may be x % above baseline or 2-3 standard deviations above baseline). The associated software application may be configured to trigger an alert upon receiving sensor data corresponding to sudden spike in pressure/force which informs a healthcare provider of this event so that the appropriate response can be provided. However to reduce the likelihood of “false positives” associated with registering pressure spikes, for example, due to general manipulation of the gel wrap 300, the software application may be calibrated according to known methods to ignore certain spikes in force/pressure readings.

Temperature sensor data received by the software application may be used to indicate to the user that the compression wrap 300 may no longer be providing the desired cooling or warming effect, likely because it has reached an equilibrium temperature with the body part or surrounding environment. In this case, it may be desirable to re-heat or re-cool the compression wrap 300. For example if the change in time of the temperature measurements shows a warming above (or a cooling below) a selected temperature threshold then it may be time to take off the wrap 300 and put it in a refrigerator so that it cools (or in a heating device so that it warms up) to a preselected temperature at which point the wrap 300 can be re-applied to the user.

In at least some embodiments, the software application may be configured with known threshold and ranges for optimal time exposure of the body part to be treated with desired pressure and temperature values.

In at least some embodiments, specialized sensors such as galvanic skin response (sweat) sensors to measure skin conductivity as an indicator of the degree of perspiration may be used.

In at least some embodiment, more advanced optical sensors such as laser Doppler blood flowmeter, for example, may be used to measure blood flow and to determine if the injured area is being targeted and treated properly. For this particular use, there may be variation in placement of the sensor such that these types of sensors are not entirely encased in gel. For example, optical sensors may be at the surface of the gel product so that they can be in direct contact with the user's skin.

In other embodiments, transducers (not shown) may be embedded for therapeutic use. For example, at least one light source may be embedded within the gel material to provide light and/or thermal therapy. For example, in some embodiments, near infrared light emitting diodes may be used as the light sources that are embedded in the gel material to induce localized tissue healing. Healing tissue in a region generally involves providing greater blood flow to the region such that there is more hemoglobin in that region. Hemoglobin has an absorption peak in the 600-700 nm range so that the amount of backscattered light from an LED is proportionately reduced by the hemoglobin. The reduced back scattering may be detected to determine the amount of hemoglobin which may indicate the amount of healing that is taking place. One or more temperature sensors may also be embedded in the gel material to monitor the degree of heating imparted on the portion of the body surrounded by the compression wrap 300.

As another example, at least one ultrasound transducer may be embedded within the gel material to provide thermal and/or vibration therapy. As another example, at least one light source and at least one ultrasound source may be embedded in the gel material to provide light, thermal and/or vibration therapy.

The sensors can be strategically placed at preselected locations along the gel band portion 320 of the compression wrap 300 to achieve improved detection of certain physiological and biomechanical parameters. Likewise, the transducers can be strategically placed at preselected locations along the gel band portion 320 of the compression wrap 300 to provide improved therapy to the user. For example, as shown in FIG. 3B, the sensors 360 and 370 may be placed proximally to the surface 330 of the gel band 320 that will be adjacent to the user's body during use to reduce the distance between the sensors 360 and 370 and the user's body when the compression wrap 300 is wrapped around the user's body to improve signal quality for signals that are measured by the sensors 360 and 370. However, the distance of the sensors 360 and 370 to the surface 330 may also be selected so that the sensors 360 and 370 are not felt by the user during use.

The embedded sensors are selected that have an operating range (as described in their published specifications) within the full manufacturing temperature range and operating temperature range under which the compression wrap 300 or other VEP products with embedded sensors (see below) are in use. For example, this may range from −20° C. (−4° F.) to +100° C. (212° F.) for everyday use. The embedded sensors may require calibration to account for temperature-dependent drift or pressure/force dependent drift to improve measurement accuracy and precision. For example, the sensors that will be embedded in a VEP gel based product described herein may be selected which have a 2.5-5.5 V DC input voltage and high accuracy and resolution (e.g. a temperature sensor with a 10 mV/° C. scale factor, ±2° C. accuracy and ±0.5° C. linearity). Technical specifications will be different, depending on the sensor and the desired biosignal accuracy.

Similar to the compression wraps 100, 200 and 200′ described in FIGS. 1A-1C or FIGS. 2A-2C, the compression wrap 300 may also be rolled into a jelly roll configuration to reduce the amount of space that it occupies during storage or during heating or freezing to a desired temperature for therapeutic use. In some embodiments, the compression wrap 300 may also be fabricated on a permanent or temporary backing member (not shown) similar to the compression wrap 200, 200′ and 200″ of FIGS. 2A-2C.

During use of any of the compression wrap embodiments described herein, the compression wrap is removed from either a warmer or cooler environment than room temperature (to allow for temperature variation within a therapeutic range as previously mentioned). It is unrolled and either removed from the storage backing material or utilized with the adhered backing material depending on the particular embodiment. At this point the user will apply one end of the wrap onto the desired body part, the user will with one hand secure the end to the body part, and then begin to surround/wrap/bandage the area while elongating the wrap beyond its resting length with the other hand. The user will continue to encompass, envelope, overlap the wrap until the entire length of the wrap is used up. Near the end of wrapping the compression wrap 100 around the user's body part, the free end of the compression wrap 100, which may be tapered or not, may be adhered to the wrap itself or may be secured by folding/tucking the fee end into one of the many folds/overlapping sections of the wrap. This process may be carried out by a secondary person to aid those that may struggle with activities of daily living.

The sensors and transducers, as well as any accompanying wires, processors and radios (all not shown) may be embedded in the gel material during the manufacture of the various polymer gel products that are described according to the teachings herein, including compression wraps, support pads, impact pads and the like. The sensors, transducers, wires, processors and radios can be referred to as electrical components. The arrangements of any of these electrical components may be achieved by placing these electrical components into a custom mold that holds the electrical components in place while heated liquid gel material is poured into the mold and cures. The gel material surrounds the electrical components as the gel material is poured into the mold and the electrical components become fully submersed into the gel. The gel material then rapidly cools and is cured and the electrical components are embedded within the gel material.

The sensors that are embedded may be small, hard sensors that are robust, withstand wear, and will resist frequent bending/movement, or flexible sensors that are designed for such movement. In some embodiments, individual sensors may be used that are wirelessly powered and wirelessly transmit data to a central terminal. In other embodiments, physical wires and connections may be used for the sensors and embedded in the gel material. For example, strategic placement of the wires within the gel material can be used such as, but not limited to, running all wires from all sensors neatly along one part of the gel product where the least amount of bending and twisting occurs. Alternatively, or in addition thereto, some embodiment may incorporate a “channel” or tunnel in the mold used to form the gel product where the channel provides for open space for the wires to run through. In some embodiments, anchors may be used in certain places. These techniques described herein help ease stress on the wires and the connections to the sensors.

These sensors can be selected based on technical specifications that meet certain needs of the gel product. For example, range, stability, noise suppression, non-linearity, resolution and sampling rate as well as cost, size, weight, temperature operating range and durability may be used as criteria for selection of sensors.

For example, some sensors may be used which have one or more specifications such as, but not limited to, (1) acceleration/motion sensitivity of 3 g, (2) force/pressure range of 100 g-10 kg range, (3) temperature resolution of about 0.06° C. with 0.5° C. accuracy in a temperature operating range of about −25° C. to +85° C., (4) size of about 10-30 mm, a small thickness of about 2-3 mm or less, (5) a weight of about 1-2 grams or less, (6) minimal processing requirements on a host processor, (7) low voltage/power requirements (e.g. 45 mW @ 3.0V), for example. In other embodiments, other ranges for these parameters may be used.

In the case of the compression wrap 300, to reduce displacement of the sensors or excessive stretching of the electrical connections, the sensors may be placed at the portion of the compression wrap 300 that is first to be wrapped on the body part and therefore closest to the surface of the compression wrap 300 which comes in contact with the skin of the body. Under these circumstances, the portion of the compression wrap 300 may be laid onto the body with minimal stretch yet obtain the desired sensing.

In the case of gel support pads, pressure sensors can be strategically placed in an array form at certain horizontal and vertical X and Y positions (but all sensors may have a systematically consistent depth location within the gel substrate). Thus, each sensor can provide an individual reading, so if one area of high pressure is measured by one of the sensors, this area can be identified (this may be accomplished via software). In some embodiments, average pressure can be measured from all pressure sensors, as well as peak pressure which is the highest measured pressure value from any one sensor. Other measures can include peak pressure area (e.g. how large of a physical area is covered by the peak pressure), overall contact area and cumulative pressure.

In some embodiments, the sensors, transducers, processors or radios may be battery powered. In other embodiments, a power connector may be made available to provide power to the electrical components requiring power. In some embodiments, rechargeable batteries may be embedded within the VEP gel material and these rechargeable batteries may be charged while the VEP gel product is not in use via a charging cable or inductive/wireless charging system.

In some embodiments, the sensors may be wireless sensors capable of transmitting data readouts wirelessly using one or more wireless communication protocols that are known to those skilled in the art. For example, the Bluetooth communication protocol may be used.

In some embodiments, polymers may be formulated to result in a VEP gel material having a selected firmness so that it may be used as a support pad. In some embodiments, the polymers may also be formulated so that the resulting VEP gel maintains sufficient flexibility to dissipate excess pressure experienced by a body part that is in contact with the support pad. In some embodiments, the gel material may be produced to attenuate vibration and/or act as a sound barrier.

In some embodiments, a support pad may be used as a mattress topper or mattress insert for assisting in the prevention of bed pressure ulcers. Accordingly, the VEP gel material, described in accordance with the teachings herein, may be produced and used for the support pad to allow the support pad to conform to the shape of the region of the body that is in contact with the support pad to dissipate the pressure at the body-pad interface. This can be done by considering various anatomical features and biomechanics of the user to select the gel formulations and contours for the end product to provide improved ergonomics for the user.

In some embodiments, the VEP gel material may be textured or molded into various conformations to permit regulation of temperature of the body part(s) in contact with the support pad. For example, in some embodiments the VEP gel material may be produced to have air pockets that may provide for cooling, air circulation and improved breathability during use.

In some embodiments, the formulation of the polymer may be adjusted to produce a VEP gel that has a selected degree of elasticity so as to permit the support pad to adjust to changes in body position.

In other embodiments, the firmness of the VEP gel material may be adjusted by altering the polymer formulation so that the VEP gel material may be used as a seat insert, seat cushion, seat back or floor mat for use in a motor vehicle for the purposes of reducing driver fatigue since the gel material may be formulated to reduce vibrations in the vehicle that would otherwise cause the vehicle operator to become fatigued or over time develop work-related cumulative trauma. For example, low level whole body vibration in the frequency range of 1-20 Hz can cause the body, spine and organs to resonate (Kitazaki, S. and Griffin. M. (1998), “Resonance behaviour of the seated human body and effects of posture”, Journal of Biomechanics, 31, 143-149; and Thalheimer, E., 1996, “Practical approach to measurement and evaluation of exposure to whole-body vibration in the workplace”, Seminars in Perinatology, 20(1), pp. 77-89). Continued exposure to low level whole body vibration may result in muscle fatigue, back pain, spinal disk degeneration, gastro-intestinal tract issues, decreased quality of sleep, headaches, circulatory issues and autonomic nervous system dysfunction (Seidel, H., 1993, “Selected health risks caused by long-term whole-body vibration”, American Journal of Industrial Medicine, 23(4), pp. 589-604; Seidel, H., 2005, “On the relationship between whole-body vibration exposure and spinal health risk”, Industrial Health 43, 361-377; and Thalheimer, E., 1996, “Practical approach to measurement and evaluation of exposure to whole-body vibration in the workplace”, Seminars in Perinatology, 20(1), pp. 77-89). The use of a VEP gel pad, in accordance with the teachings herein, may dissipate these low level frequencies. When measuring whole body vibration exposure in the workplace, the ISO 2631-1 standards (i.e. Mechanical vibration and shock—Evaluation of human exposure to whole-body vibration, Part 1—General requirements, International Organization for Standardization, Switzerland, 1977) are the most widely accepted (with various updates since). The frequency of exposure may be compared with daily exposure limits that have been established in the literature, such as, but not limited to, the ISO 2631-1 guidelines, to better guide prevention of cumulative injury. In accordance with the teachings herein, VEP gel pads with embedded sensors can provide real time data monitoring and analysis to determine the cumulative effects of the vibration exposure over time to determine whether or not an individual's exposure has reached health guidance caution zones. For example, when using a gel pad for a hospital bed, pressure/force measurements can be used to monitor vibrations. Alternatively, for automotive applications such as trucking, or for hand arm vibration tool use, a high-resolution accelerometer can be embedded into the gel pad to measure vibration since some pressure/force sensors may not have the frequency range or sampling rates needed for these applications.

In addition, for long distance travel, an in-vehicle VEP gel mattress, in accordance with the teachings herein, may be provided which minimizes sleep ‘disruptions’ thereby allowing the operator to have good quality sleep and less fatigue when awake. In some embodiments, the driving and sleeping situations can be monitored by using sensors embedded in the VEP gel material to determine if the operator is becoming more ‘vulnerable’ to fatigue (e.g. due to accumulated vibrations) and real time results can be available to the operator as well as a central monitor, such as a dispatcher.

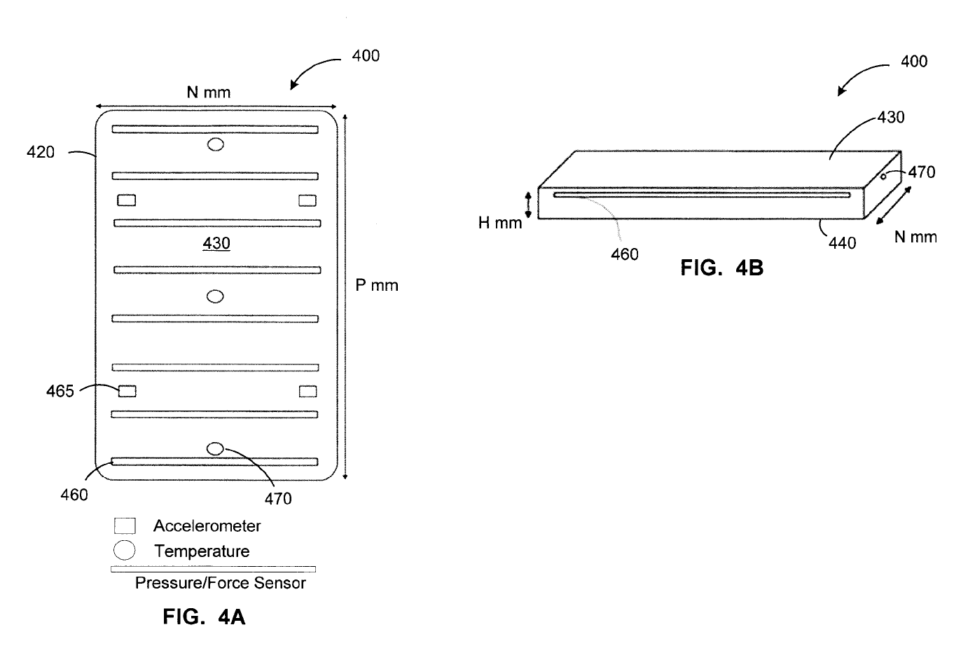

Referring now to FIGS. 4A-4B, shown therein are various views of a VEP gel support pad 400 made using a gel material 420 with embedded sensors 460, 465 and 470 in accordance with the teachings herein. The gel support pad 400 has a selected firmness and thickness that can be achieved by the manufacturing process described in accordance with the teachings herein so that the support pad 400 improves at least one of ease of use, comfort and quality. The embedded sensors 460, 465 and 470 are configured to monitor one or more physiological and biomechanical parameters. In this example, the sensor 460 is a pressure/force sensor, the sensor 465 is an accelerometer and the sensor 470 is a temperature sensor. In general, the embedded sensors can have many different shapes and sizes which can be selected based on the particular VEP based product or particular application thereof. For example, some embodiments of these VEP based products can have a variety of physiological sensors in unique locations.

The location of a particular sensor can be selected based on the nature of the measurements that are being made. For example, for VEP pads used in beds in hospitals and care homes, an array of pressure sensors may be used throughout the entire VEP gel pad. The sensors that are used may have greater sensitivity in certain areas of the VEP gel pad that correspond to whether the user's heels (calcaneus), hips (ischial tuberosity), elbows, shoulders and back of their head are in constant contact with the surface of the VEP gel pad. Higher rates of pressure ulcer development is likely at these anatomical sites. For example, an array of pressure sensors may be used that has a resolution of 1-2 cm² for sensing area and cover certain portions of the bed, such as the example locations just given. Accelerometers and temperature sensors may be placed in a few locations such as the top, middle and bottom of the VEP pad.

Furthermore, in at least some embodiments of the support pad 400, surface texturing consistent with various types of molds may be used to shape the designs to enable the VEP pad 400 to be more effective in reducing body contact issues such as one or more of friction, shear, heat build-up, moisture and pressure, for example. Surface texturing may include using various channels in the surface of the VEP product (e.g. pad, wrap, etc.) where the channels can have certain designs and be in certain patterns such as chevrons (e.g. upside down V shapes), rows or columns of lines or a combination of chevrons and rows or columns of lines. These channels may also be referred to as air pockets. The decision process in selecting the appropriate gel formula, for a desired end product, is the same as those factors used for the compression wrap.

The thickness of the support pad 400 may range from one half to one inch to provide sufficient dissipation of pressure (e.g. “localized point pressure”) between the body and the support pad 400 to maximize comfort of the user, who may be resting or sleeping. Furthermore, since the sensors 460, 465 and 470 and other electrical components are embedded within the gel material, the gel material can protect the electrical components from physical impact due to the pressure dissipation characteristics of the gel material and may also increase the useful lifetime of the electrical components since the physical characteristics of the gel material (e.g. flexibility and elasticity) can be selected to withstand normal wear and tear for a long term period based on the gel formulation that is selected.

A number of different sensors may be embedded throughout the support pad 400 to measure physiological and biomechanical parameters of the user. The sensors may include any combination of pressure/force sensors 460, accelerometers 465, and temperature sensors 470. The sensors 460, 465 and 470 may be embedded in to the gel material 420 proximal to the upper surface 430 or lower surface 440 of the gel material 420.

In embodiments which include the accelerometers 465, the accelerometers 465 may be used to track the user's movement in three dimensions, for example, as the user shifts from one resting/sitting position to another. In some applications, accelerometers may be used to measure and track vibrations. For example, for whole body body vibration measurement, measured accelerometer data can be processed as per ISO standards. The measured data can be filtered, and certain analysis techniques, such as the frequency weighted root mean square for each axis of vibration, can be determined and compared against exposure limits for cumulative vibration found in the ISO 2631 standard. For other applications such as using the gel pads to reduce and/or monitor hand-arm vibrations, the measured vibrations can be determined and compared to the ISO 5349 (2001) standard.

The arrangement and placement of the various sensors may be predetermined to improve the measurement of parameters of interest while maintaining comfort of the user of the support pad 400. Furthermore, in some embodiments, different sensor locations and/or types of sensors may be used with particular gel formulations to provide more effective (and accurate) monitoring depending on what area of the body is to be monitored and for what purpose. For example, a VEP support pad designed for a user to sit on in an upright position may have more sensors placed in known body contact points or areas known to be subject to high pressure. Identification of these locations for the VEP gel pad requires consideration of user anatomy and body anthropometry for individuals of various sizes. Therefore, in some embodiments, the VEP support pad 400 may be available in different sizes (e.g. small, medium, large etc.) to accommodate users of different body size.

As another example, where the VEP support pad 400 is used as a sleep support or mattress, the type of measurement sensor may determine the sensor's placement along the pad. For instance, heart rate sensors and accelerometers may be placed closer to locations of the VEP support pad 400 that are more likely to be in contact with the user's body where those measurements should be made such as the user's upper torso, while temperature sensors may be placed at a different location.

In any of the VEP gel products described in accordance with the teachings herein, for optimal measurement sensitivity, the sensors may be placed proximal to the surface of the VEP support pad 400 that is in contact with the user's body, but not too close to the surface to cause user discomfort. In some embodiments, where the polymer formulation results in a softer VEP gel material, higher signal amplification or sensors capable of outputting data at higher resolution may be required to accommodate additional user movement transferred to the sensors through the softer VEP gel during use.

During the manufacturing process, as explained previously, a customized mold may be used to assist in keeping the sensors in place as the liquid VEP gel material is poured into the mold and surrounds the sensors such that the sensors are embedded within the VEP gel material when the VEP gel material is cured.

In some embodiments, the support pad 400 may be used as a part of a sleep monitoring system which unobtrusively collects data captured by the sensors within the support pad 400 to develop a sleep profile for a user of the support pad 400 to help identify and correct possible causes of sleeping disorders for the user such as, but not limited to, sleep apnea. The parameters that can be measured may include peak pressure, average pressure, cumulative pressure and pressure area of the user's body on the support pad 400 as well as the average temperature of the support pad 400. Through the use of the VEP gel manufacturing process described herein, the VEP gel material can be formulated to have a certain selected thermal conductivity such that the average temperature of the support pad 400 may be generally maintained several degrees cooler than the body temperature to facilitate more restful sleep. Thermal testing may be done on different gel compositions to determine their thermal coefficients to allow for a gel formulation that has an appropriate thermal coefficient to be used.

In some embodiments, the support pad 400 may be configured to minimize sleep-disrupting factors such as excessive body point pressure or excess temperature build-up and pressure ulcers that are problematic with conventional sleep supports, such as foam mattresses, so as to ensure that the parameters measured by the sensors in the support pad 400 are more likely to indicate personal sleep issues rather than those caused by the conventional sleep support. For example, test results on different support pads were conducted and indicated that different sleep support materials, i.e. foam, gel and various combinations thereof, have different pressure dissipating capabilities and different surface designs (i.e. surface texture) also contribute to different pressure dissipating capabilities and that certain combinations of materials and surface designs can be used so that the parameters measured by the sensors in the support pad 400 are more likely to indicate personal sleep issues rather than those caused by the conventional sleep support. Furthermore, as the VEP gel material can be made to moderate the user's temperature and dissipate any pressure points, the user's comfort and sleep quality will increase and any measured data will indicate average pressure/temperature no matter where a pressure point is in relation to a sensor location.

In some embodiments, sensed sleep data may be logged (i.e. “journaled”) by the sleep monitoring system and be used to develop a correlation between the user's perceived sleep quality (e.g. as provided by the user) with the user's movements as measured by the embedded accelerometers while the user is asleep. Supplemental data may be obtained using other sensors such as heart rate sensors to determine heart rate variability and ratios of beat intervals and motion sensors to monitor a user's sleep stages, such as when the user enters restful sleep versus light sleep, in terms of length, quality of sleep and how many times the user wakes up. In some embodiments, the journaled data may be used to build a user “sleep profile” based on physical attributes like weight, physical size, heart rate, and patterns of movement, so that a sleep monitoring system may distinguish one sleeper from another without the sleeper having to identify themselves.

It should be noted that there may be some embodiments in which the support pad 400 does not include electrical components.

It should be noted that in some embodiments the support pad 400 includes transducers and/or electrical components as was described for the compression wrap 300 to provide therapeutic benefits.

It should be noted that in some embodiments of the various polymer gel products described in accordance with the teachings herein, the VEP gel material may be selected and made so that the VEP gel material will not totally harden when it is cooled (i.e. it is semi-solid), so that the VEP gel material is very durable and/or so that the VEP gel material acts as a contaminant barrier.

It should be noted that FIG. 4B can be considered to show sensors 460 and 470 oriented at the edges of the support pad 400. Alternatively, FIG. 4B can be considered to show slices along the side and top edges of the support pad 400 showing the location of the sensors 460 and 470 embedded within the gel layer of the support pad 400.

It should be noted that in the embodiments of the various polymer gel products described in accordance with the teachings herein which have embedded electrical components, the VEP gel material provides protection to the electrical components.

Also, since the VEP gel material may be made in a repetitive and consistent manner, embedded sensors in two separate polymer gel products will provide consistent real-time data for the same user and the data can be calibrated. This is in contrast to other sensors that may be otherwise fastened to a surface of a material in which the sensor position may move or it may be damaged and not be able to sense information consistently.

It should be noted that for at least some of the various embodiments of the polymer gel products described in accordance with the teachings herein the gel material may be selected and the products made with an impermeable surface thereby allowing the polymer gel products to be cleaned and disinfected, without being damaged. For example, the polymer gel products may be washed and disinfected with household or medical grade cleaning products and then reused.

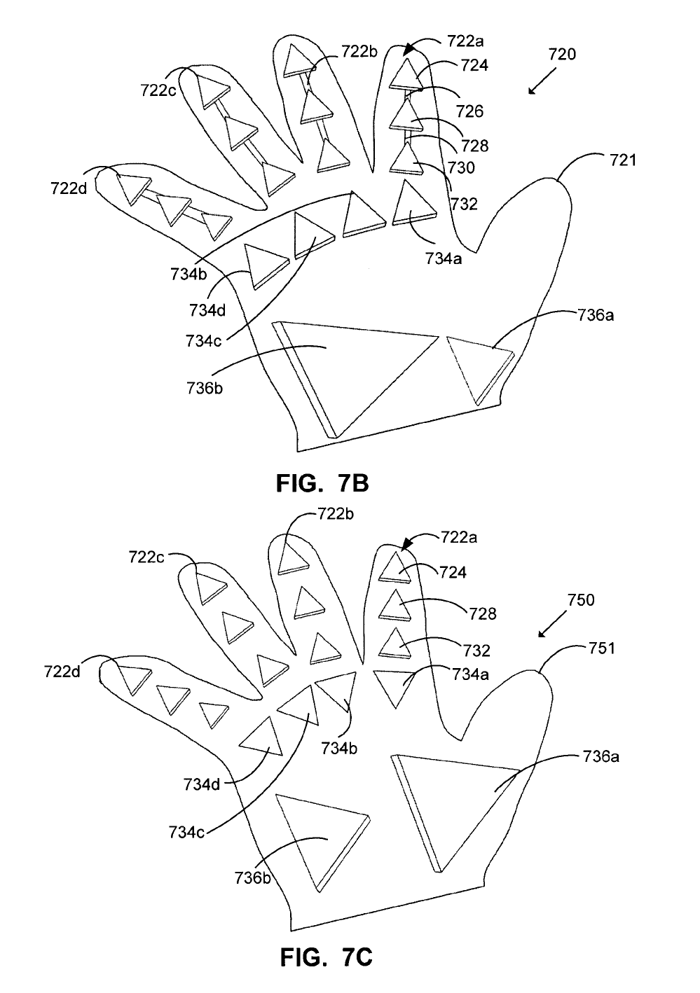

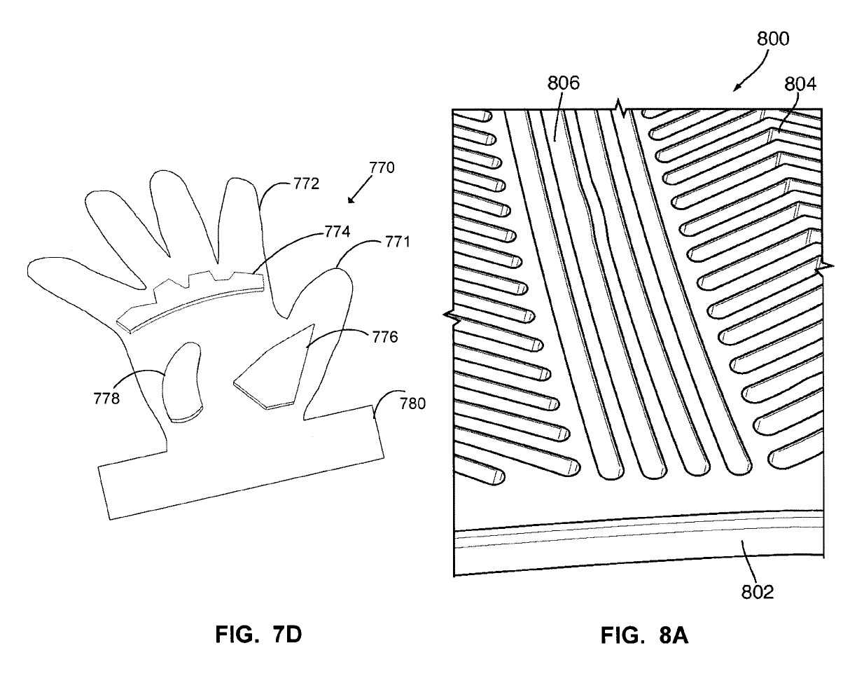

There are a variety of different polymer gel products that may be made in accordance with the teachings herein. For example, the support pads described herein with or without embedded electronics may be configured for use as a mattress topper (e.g. for placement on top of a current mattress), as a mattress insert, as a seat cushion pad, as a seat insert pad, as a seat back support pad, as a floor mat, as a bed mat, as a body pad (e.g. to provide comfort to the body and/or head), and as an impact or shock dissipating pad (e.g. helmet, shoulder pads, gloves etc. to provide protection to a user's corresponding body part). For example, for helmets a VEP gel insert pad with embedded electronics may be inserted into the helmet to protect the wearer and also provide force and acceleration measurements to determine if the wearer may be susceptible to a concussion due to an impact. After impact (i.e. deformation due to an applied force) the VEP gel pad may return to its original shape to dissipate any subsequent impacts. As another example, the compression wrap may be modified for use as an anti-vibration glove (see FIGS. 7A-7D for example embodiments) or anti-vibration sock.

It should be that the various polymer gel products described in accordance with the teachings herein may be used in a variety of different settings and for various purposes. For example, the polymer gel support pads with embedded sensors may be used by sleep professionals to assist in making diagnoses of their patients, and/or by sleep researchers to improve test and research procedures. In fact, sleep labs can leverage these polymer gel pads in portable sleep technology to expand their services to various markets such as for shift workers and/or commercial drivers as well as to allow for home testing of patients where they are more likely to sleep as they normally do allowing for more accurate sleep tests to be performed. The polymer gel products may also be used by seniors living in their own home, a retirement home or a long-term care facility where monitoring can take place nightly while in the person's usual bed and bedroom and polymer gel sleep pads may be used to prevent the occurrence of bed sores/ulcers. Hospitals may also use these polymer gel products described herein to improve patient monitoring and data collection which may improve care, and reduce costs.

Formulating a VEP gel requires knowledge and understanding of the primary characteristics of each ingredient and the relationship between them when combined in a given formulation. For example, one or more polymers having certain molecular weights may be combined with mineral oils of various viscosities in a desired ratio to obtain a mixture or slurry which is processed to produce the gel material. The molecular weights of the polymers, the viscosity of the mineral oils and the ratio of these ingredients are selected to produce a gel material having desired values for one or more desired physical characteristics such as softness, tackiness (e.g. a degree of stickiness or adhesion), elongation, tensile strength and thermal conductivity.

In some cases, vegetable oils may be used instead of mineral oils. Vegetable oil is more environmentally friendly and is renewable.

Generally, high molecular weight polymers provide a higher tensile strength, a higher tear strength, a higher melt viscosity, a higher softening point, a higher heat-distortion temperature and a higher processing temperature. Higher molecular weight polymers also result in gel materials that are more resilient. Additionally, polymers with increasing molecular weights generally have decreasing melt flow (e.g. the viscosity of a melted material), and decreasing melt processability, which means a reduced ease of mold filling, and reduced ability to cast or mold gel materials to have thin cross sections.

Mineral oils used for the preparation of the VEP gel material may be obtained from industrial or retail sources. Some types of mineral oils, such as white mineral oil, may be suitable for use in making VEP gel material for human use since the oil itself may be used to treat topical conditions such as dry or irritated skin. The viscosities of mineral oils may be classified according to grade such as medium grade or light grade oils. Generally, oil grades affect the resilience or the “bounce” properties of the resultant gel material and also the absorbance of the gel material. For example, use of a light grade mineral oil results in a gel material that provides greater wicking and has higher resilience. A less resilient gel is more “sticky”. In addition, use of a lighter oil will penetrate the polymer faster at a given temperature allowing for reduced processing times although the oil-polymer mixture may be left to stand for a minimum period of time before further processing to allow saturation of the polymers by the oil products. However, a less viscous oil will have a higher melt flow and melt processability and a lower heat distortion/softening point for any given polymer/oil blend.

In general, oil to polymer ratios ranging from 4:1 to 20:1 may be used, depending on the desired characteristics of the finished gel based product. The selection of the grade of the oil and the particular polymers, again, depends on the desired (i.e. selected) characteristics for the finished gel based product. For example, the finished gel based product becomes more stretchy (i.e. more elastic) and less firm as the oil to polymer ratio increases.

In some embodiments, a stabilizer may be added to the polymer-oil mix to maintain certain physical characteristics for the resulting gel material. For example, the stabilizer may be an antioxidant that is used for organic substrates to protect against thermo-oxidative degradation. Stabilizers may generally be added in the range of 1% to 5% of the amount of polymer that is used.

Polymer-based slurries require time to set or polymerize to form a gel, which permits the polymer to be manipulated prior to polymerization to obtain a VEP gel having a desired shape. Extrusion and molding techniques may be used to determine the shape of the resultant VEP gel material. For example various molds may be used to provide the resultant VEP gel material with different textures, shapes and sizes that may further enhance the functionality of the VEP gel material when used in one of the polymer gel products described in accordance with the teachings herein.

For example, in some of the embodiments of the polymer gel products described herein, a selected polymer formulation may be used to produce a VEP gel that is firm and capable of providing a desired (i.e. selected) level of support (e.g. firmness). In some embodiments, such a VEP gel may also be shaped to form a VEP gel pad or a VEP gel support (described in detail below) for pressure dissipating applications. In other embodiments, the polymer formulation may be selected to produce a highly elastic and flexible gel so that it may be used as a VEP gel band (described in detail below) for the application of localized pressure.

In yet other embodiments, various additives may be mixed into the polymer formulation prior to polymerization so that the resultant VEP gel material has certain desired physical qualities such as, but not limited to one or more of a desired colour, a desired scent, desired antimicrobial properties, and desired fire retardant properties. For example, if the gel material is to have a blue color, then blue pigment is added. Likewise if a yellow color is desired, then yellow pigment can be added. Generally, the additive has to be able to withstand the maximum processing temperature (as described below) and be non-flammable and non-toxic. Additives may be added in the ml range such as, but not limited to, from 1 ml to 15 ml, or tens of ml, for example.

Referring now to FIG. 5, shown therein is a block diagram of an example embodiment of a processing line 500 that is used to produce polymer gel products in accordance with the teachings herein. The processing line 500 generally comprises a mixing stage 502, an extrusion stage 504, a material processing stage 506 and a casting stage 508. The mixing stage 205 generally comprises a mixer 510 and a heater 512. The extrusion stage 504 generally comprises a feed zone 514, a core zone 516 and an output zone 518. The material processing stage 506 generally comprises a spreader bar 520 and a conveyor 522. The casting stage 508 generally comprises a dispenser 524 and molds 526. It should be noted that there may be other embodiments of the processing line 500 that may have different elements depending on the particular type of polymer gel product that is being made.

The component materials used to fabricate the VEP gel material (e.g. a formulation of polymers, mineral oils, stabilizers and/or additives) for the polymer gel products are provided to the mixer 510 where they are mixed into a slurry. For example, several gallons of materials can be provided to a hopper which then feeds the materials to the mixer 510. The slurry is then provided to the heater 512 and the slurry is heated to a desired temperature between 191° C.-213° C. (375° F.-415° F.). The temperature is determined based on the viscosity of the mineral oil and the selected polymer formulation. For example, a lower viscosity is associated with a thinner oil, which means a lower processing temperature is used. In contrast, a higher viscosity is associated with a thicker oil, which means a higher processing temperature is used.

Various types of polymers from various suppliers may be used in the polymer formulation such as, but not limited to, those available from Northstar Polymers or MacDavid Wellness Solutions, for example. In some cases, additives to provide additional physical characteristics to the resultant gel may also be incorporated into the slurry at the mixer 510. A range of polymer formulations are possible and a particular formulation may be selected to achieve a certain performance or set of characteristics. For example, the polymer formulation may be selected based on the resulting VEP gel material having, but not limited to, one or more of: a desired (i.e. selected) degree of impact protection, temperature regulation, lifetime, anti-microbial properties, environmentally friendly characteristics, non-allergenic characteristics, colour, and scent, for example. The slurry may be processed by the mixer after 24 hours, the time being dependent on the saturation point of the selected polymer formulation in the slurry.

The particular polymer formulation that is selected and the processing of the polymer formulation may also depend on the size and nature of the polymer gel product that is being made.

The heated slurry is then processed by the extrusion stage 504. The extruder speed, operating temperature of the extrusion zones and the output rate may be varied depending on the composition of the slurry and the desired (i.e. selected) properties of the resulting gel material. For example, the length of time that the slurry is processed in the extrusion stage 504 may depend on the thickness of the slurry with thicker slurries taking longer to process.

The heated slurry is introduced into the feed zone 514 of the extrusion stage 504. For example, the slurry may be introduced to a hopper which sends the slurry to an input area of a rotating screw in the feed zone 514. The slurry then flows into the core zone 516, in which the slurry is pulled along a barrel by a rotating screw (not shown) that is within the barrel. The slurry travels along the length of the screw. The barrel may be heated by heaters that are disposed externally around the barrel portion of the screw so that the core zone 516 of the extruder is heated to a temperature suitable for the particular slurry formulation that is being processed. The diameter of the screw may increase along the barrel portion so that the slurry is compressed which results in friction and heating of the slurry as well as further mixing of the slurry ingredients. The slurry then reaches the end of the screw where it is referred to as a slurry melt that is at a certain desirable (i.e. selected) pressure and temperature for further processing. The slurry melt enters the output zone 518 of the extruder and exits as a gel. In some embodiments, a screen pack may be located at the output zone 518 to filter out any impurities or contaminants from the gel. The gel may be inspected at this point for quality control.

In some embodiments, the temperatures of the feed zone 514, the core zone 516 and the output zone 518 of the extrusion stage 504 may be the same. In other embodiments, the temperatures of these zones may be different. For example, the temperature of the core zone 516 may be set to be higher than the temperature of the feed zone 514. The temperature variation between the feed zone 514, the core zone 516 and the output zone 518 may be used to control the extruder speed, that is, the speed of the flow rate of the slurry from the feed zone 514, through the barrel (i.e. barrel speed) in the core zone 516, and through the output zone 518 (i.e. output rate). All of the temperatures (e.g. 3) are selected based on the formulation of the slurry as well as the density, thickness and viscosity of the slurry and may generally be between 191-213° C. (375-415° F.), for example. However, generally, the temperature in the different zones follows the pattern whereby the highest temperature point is in the core zone. For example, if the highest temperature is 204° C. (400° F.), then the temperatures from the first zone 514 to the last zone 518 may be 191° C. (375° F.), 204° C. (400° F.) and 199° C. (390° F.), respectively. As another example, if the maximum processing temperature is 199° C. (390° F.), then the temperatures may be 185° C. (365° F.), 199° C. (390° F.) and 193° C. (380° F.), from the first zone 514 to the last zone 518, respectively.

The gel is then processed by the material processing stage 506 to allow the extruded slurry to be distributed by a spreader bar 520 onto the conveyor 522. The material processing stage 506 includes a dispenser which receives the hot polymer liquid gel from the extruder and dispenses the polymer liquid gel through a nozzle to the spreader bar 520. The volume of heated gel liquid that is dispensed by the nozzle is controlled by a trigger. A hose connects the nozzle to the spreader bar 520. The hose may be heated to keep the liquid gel at an appropriate temperature for material processing.

In some embodiments, a tap flow nozzle may be used to allow the liquid gel to be dispensed while holding the nozzle in one's hand and increasing or decreasing the flow rate of the liquid gel. In other embodiments, an open nozzle may be used which has a fixed output location and the mold, may be placed on the pouring table (e.g. conveyor 522), and moved to a filling location under the open nozzle. The tap flow nozzle may be held in an operator's hand, which allows for a number of molds to be placed on a surface and the nozzle moved to fill the molds, rather than the molds/table being moved in order to fill the molds.

The spreader bar 520 acts as a pouring component having a desired width and a desired number of holes to permit distribution of the liquid gel material on the conveyor 522. The diameter of the holes and the pitch between the holes may be selected based on the viscosity of the liquid gel material that is being processed. The pattern of the holes may also be selected depending on the number of molds 526 that are being used so that several polymer gel products can be formed at the same time. For example, if the holes were too wide apart, then the poured liquid gel may not flow together to form one layer; if the holes were too close then the liquid gel may flow together from the holes and the layer of poured liquid gel may be too thick or the liquid gel may be too thick in the middle of the spreader bar 514 and not reach the ends of the spreader bar 514; if the holes are too large then the liquid gel may not reach the ends of spreader bar 514 and if the holes are too small, then the poured liquid gel may not reach the edges of the mold.

The conveyor 522 may be heated if the ambient temperature will lead to premature cooling and premature curing of the liquid gel material. For example, the conveyor 522 (which may be a pouring table in some embodiments) may be at a temperature in the range of about 38° C.-66° C. (100° F.-150° F.). The temperature depends on a number of factors such as ambient temperature, mold material, and temperature of the mold. For example, as the mold use increases, the mold retains heats and its temperature increases, so the temperature of the conveyor 522 may be decreased. The conveyor 522 may be preheated before the slurry starts being processed.

The spreader bar 514 may also be heated (as described below) to maintain a desired gel pourability and gel viscosity so as to permit the liquid gel to be sent to the casting stage 508 at a desired rate. For example, heating strips may be affixed to the spreader bar 514 and a thermostat may be used to adjust the temperature on the surface of the spreader bar 514.

Since the liquid gel is dispensed by the spreader bar 520 onto the conveyor 522, the height or distance of the spreader bar 520 relative to the conveyor 522 may also be a relevant consideration. For example, if the spreader bar 520 is too high (e.g. in some cases just even 3 inches from the conveyor surface 522) then the liquid gel may cool too much as it is dispensed and it will not be possible to uniformly distribute the liquid gel into a mold thereby adversely affecting the quality of the resulting polymer gel product. The spreader bar 514 may also be temperature regulated in some embodiments so as to facilitate the flow of the liquid gel material. If the dispensed liquid gel is below a certain flow temperature (depends on gel formulation), then it won't pour, fill and cure as expected. Conversely, if the temperature is too high, then this may negatively impact on the mold (depending on the mold material) as the liquid gel that is too hot may discolour and/or misshape the mold. The spreader bar 514 may also be configured in some embodiments to evenly distribute the liquid gel material so that it can be applied to the entire width of a mold.

In the casting stage 508, the liquid gel from the conveyor 522 may be dispensed through the dispenser 524 into one or more molds 526 at a high temperature. In some instances the liquid gel may be dispensed onto a flat surface upon which a VEP gel sheet may be produced for a polymer gel product. In other instances, the liquid gel slurry may be dispensed into one or more molds 526 of various shapes. The shape, size and number of molds 526 depend on the polymer gel product being made. For example, the molds may be small such as on the order of a tray size, or the molds may be larger such as about 29″×15″×1½″ (73.66 cm×38.1 cm×3.81 cm) or the molds may have non-rectangular shapes such as rings within a rectangular mold of about 19″×19″×1½″ (48.26 cm×48.26 cm×3.81 cm).

In some embodiments, two or more liquid gel formulations can be poured (e.g. layered) into the same mold to produce a desired VEP gel material with composite or combined properties which may be advantageous for certain polymer gel products. For example, one gel formulation that is very elastic can be used to provide one layer of the gel product and another gel formulation that is elastic can be used to provide another layer of the gel product to result in an end product which incorporates the characteristics of both. As another example, two gel formulations may be similarly layered to result in a polymer gel product that has a sticky surface due to the layer of first gel formulation and an opposing less sticky/adhesive surface due to the layer of the second gel formulation. As another example, two gel formulations may be used to result in a polymer gel product that has different colors on opposing surfaces, or different scents on opposing surfaces.

In some embodiments, the outflow of the liquid gel into the molds 526 may be through an open-hole dispenser, providing unrestricted output. In other embodiments, the outflow may be constrained through a nozzle which may “tap” the flow. In yet other embodiments, the liquid gel may be distributed into a heated holding tank in which the slurry may be dispensed through a hose or a pistol.

The material used for the molds 520 may include, but are not limited to, silicone, copper, cast iron, Teflon coated surfaces, aluminum, tin foil, ceramic, rubber, polyethylene terephthalate or polycarbonate, for example. The particular material that is used for the mold may depend on the type of polymer gel product that is being formed as well as the manufacturing cost. For example, the mold may be made using a metal frame, which allows for flexibility as steel strips of various dimensions are readily available allowing for molds of various dimensions for the base and height. As another example, molds made of silicone are very resilient and cost-effective. Tin foil molds are also cost-effective, but are for one-time use. Cast iron molds are very effective as well and durable but a cool down period is used before new liquid gel may be poured into the cast iron mold.

Once the liquid VEP gel is dispensed within a mold, the liquid VEP gel is then allowed to polymerize or “set” over time to form one of the solid polymer gel products described in accordance with the teachings herein. After setting, the resultant VEP gel product is then removed from the mold 520. The setting or curing time required generally depends on the formulation of the liquid gel, the temperature of the liquid gel at the time it was dispensed into the molds 520, and the ambient temperature surrounding the liquid gel.

The various polymer gel products that may be produced using the processing line 500 may need different materials and operating parameters. Various examples are provided below for a polymer gel seat cushion, an impact polymer gel pad, polymer gel products with embedded sensors, a polymer gel pressure pad and various gel surface finishes. Other types of gel-based products can also be made using the teachings herein.

Referring now to FIG. 6, shown therein is a flow chart of an example embodiment of a method 600 for producing polymer gel products in accordance with the teachings herein.

At 602, polymers of various molecular weights and mineral oils of various viscosities may be combined in the desired (i.e. selected) ratios to obtain a gel having certain values or ranges of values for certain desired (i.e. selected) physical traits such as, but not limited to, softness, tackiness (i.e. stickiness or degree of adhesion), elongation, and tensile strength, for example. Consequently, the formulation options based on polymer molecular weights and oil viscosities may cover an extremely wide range of physical characteristics. As discussed previously, high molecular weight polymers generally provide higher tensile strength, higher tear strength, higher melt viscosity, higher softening point, higher heat-distortion temperature and higher processing temperature. Additionally, the greater molecular weight polymer generally decreases melt flow and decreases melt processability. These characteristics facilitate ease of mold filling and the ability to cast or mold gel materials with thin cross sections. The viscosities of mineral oils may be classified under various grades such as medium or light grade oil. Generally, oil grades affect the resilience or the “bounce” properties of the resultant gel material. For example, light grade mineral oil is known to increase the wicking ability feature and increase the elasticity of the resultant gel material.

At 604, in some embodiments, the slurry may be processed, for instance, by heating the mixture to a desired temperature to attain a specific viscosity to allow for certain manipulations to be performed. The processing may involve passing the slurry through the processing line 500 as described in FIG. 5 or through another suitable processing line. In some cases, additives and/or stabilizers may be introduced in the processing and mixed into the polymer-oil solution prior to polymerization to give the resultant gel material different physical qualities such as certain color, scent, antimicrobial properties, and/or fire retardant properties. Examples of additives include pigments, natural oils for aroma and sparkles for ornamental design of the finished gel product.

The slurry is processed by an extruder to form a liquid VEP gel which is then dispensed into one or more molds at 606, depending on the shape and size of the polymer gel products that are being made. Once the liquid VEP gel is poured into the mold(s) it is allowed to polymerize or “set” to form a solid VEP gel. Molds of various shapes and materials may be used to impart different physical characteristics to the VEP gel material. For example, textured molds may be used to impart one or more textured surfaces to the VEP gel material. In some other instances, no mold is used. Instead, the slurry may be dispensed onto a flat surface to create a VEP gel sheet. The VEP gel sheet may be cut into various shapes and sizes, where some of the sheets may be used to create VEP gel bands or VEP gel support pads having elastic properties or gel pads having certain support and pressure dissipation properties, for example. At 608, the VEP gel product is removed from the mold and the process may be repeated to create more polymer gel products.

### Example 1: Polymer Gel Seat Cushion

For a polymer gel seat cushion, there are various parameters that may be considered during manufacture such as: (1) selecting a gel formulation and thickness to obtain certain values for elasticity and shear reduction as well as possibly selecting a textured surface; (2) selecting a cradle durability to dampen acceleration (i.e. vibration) encountered by the user (along the x and y axis support); (3) selecting a type and thickness of foam material upon which the VEP gel material is placed as an upper layer or within which the VEP gel material is placed as an insert (various foam grades may be used in order to craft a suitable final product and injected foam may be used in a conventional manner or in a new manner as described below); and (4) selecting encasement materials to house the gel material and foam material (certain textiles may be used).

When using a conventional foam injection technique, the gel layer and the foam layer are formed in two steps. For example, conventional injected foam products may be made one at a time by injecting a foam mixture (e.g. isocyanates, polyols, catalysts and additives) into a mold. Once the foam mixture is injected into the mold there is a chemical reaction that results in the foam mixture rising and expanding to then occupy the whole space in the mold. The mold contents then solidify and the resulting product can then be removed from the mold either mechanically or manually. A gel layer can then be added to the already manufactured injected foam product. The gel layer may be attached to the foam by an adhesive or may be unfixed.

Alternatively, in accordance with the teachings herein, the gel based product can be made by forming a VEP gel layer around which foam can be formed in an integral process. For example, a layer of gel material can be placed in an injected foam mold prior to the injection of the foam mixture. Once the foam mixture is injected, and rises and expands to fill the mold, the gel layer becomes an integral part of the final solidified product. The newly formed foam frames the gel layer on the surface and the stickiness of the back surface/layer of the gel adheres to the underlying foam.

In the case of gel-based products that incorporate embedded sensors, the sensors and other electronic circuitry can be embedded in the gel layer as described herein and then the gel layer with the embedded sensors can be placed in the injected foam mold and the foam mixture injected as described above to form the final solidified product.

Gel based products formed using this integral process can provide important personal wellness benefits (these gel based products may also be referred to as gel injected foam products). For example, an injected foam mold is usually contoured so as to enable the final product to fit and provide enhanced support and comfort to a user (for example, the final product may be a cushion). With the gel injected foam products, because of the rise and expand aspect of the injected foam process, the gel layer will also be shaped during the injection process to reflect the precise contours of the mold and will be permanently attached to the foam by adhesion. The gel layer may be on an upper surface or lower surface of the foam layer. This gel layer will not only protect the foam from contaminants but will also ensure that the full benefit of the gel's ability to dissipate pressure and temperature will be realized in the performance of the final product.

In some embodiments, the gel layer may have one or more irregular surfaces which will enable the gel injected foam products to better meet performance requirements. The foam injection process will accommodate the irregular shaped gel layer and result in a void free foam attachment with the gel layer. Such gel injected foam products can provide customized support/comfort products to satisfy particular personal wellness diagnosed needs.

It should be noted that the gel injected foam products can be a cushion, a seat, a support pad, a back support, or other body part support such as an arm rest, for example.

### Example 2: Impact Polymer Gel Pad

For an impact polymer gel pad to be used in body pads, gloves or sports helmets, there are various parameters that may be considered during manufacture such as: (1) selecting a gel formulation and a gel pouring method; (2) researching formulation additives (e.g. nano powders, glass micro bubbles, etc.); (3) pouring gel samples with additives; (4) creating gel based samples for impact testing; and (5) testing gel samples for impact, weight savings, and temperature dissipation.

Referring now to FIGS. 7A-7D, shown therein are images of various example embodiments of anti-vibration gloves (or vibration reducing gloves) or parts of these gloves with gel pads for reducing or absorbing vibrations in accordance with the teachings herein. These gloves can be worn by a user who touches objects that vibrate or receive an impact, such as golf clubs, hockey sticks, baseball bats, and other sports equipment or steering wheels, drills, jackhammers or other vehicle, manufacturing or industrial equipment. The gel pads used for the gloves 700, 720, 750 and 770 may be disposed at one or more of the lower surface of the glove's fingers, the upper palm surface of the glove and the lower palm surface of the glove. In alternative embodiments, there can be gel pads at the thumb regions of the gloves shown herein.

It should be noted that there may be other embodiments where the gel pads are incorporated into other wearable articles such as socks with gel pads disposed at one or more of the heal, toe and arch regions. In another alternative embodiment, gel pads can be incorporated into the knee regions of pants used by manual laborers who kneel while the work (such as ceramic tile or floor installers). In another alternative embodiment, gel pads can be incorporated into the hip regions of pants to help prevent hip fractures if the pant wearer falls on their side.

The various gel pads used for gloves 700, 720, 750 and 770 can be made in accordance with the teachings herein. In some embodiments, at least one of the gel pads of the gloves 700, 720, 750 and 770 has a textured surface, such as one of the textured surfaces described herein for the other gel based products, to further reduce the impact of vibrations on the hand of the user wearing one of gloves 700, 720, 750 and 770. Alternatively, in some embodiments, only one or more of the gel pads on the lower palm surface of the gloves 700, 720, 750 and 770 may have a textured surface as described herein for the other gel based products.

One or more of the various gel pads can be located on, attached to or adhered to an outer surface of one or more of gloves 700, 720, 750 and 770. In an alternative embodiment, one or more of the various gel pads of one or more of gloves 700, 720, 750 and 770 can be covered by the material used to make the glove, such as cloth, nylon or another suitable material, such that these covered gel pads are not visible and/or are protected from the elements to improve their durability and longevity. The gel material composition, thickness and shape may be selected based on the type of work that the glove is being used for and therefore the amount of vibration that the glove is made to attenuate.

Referring now to FIG. 7A, shown therein is a glove 700 with an outer surface 701 having a single upper palm gel pad 702 and two lower palm gel pads 704 and 706. There is no complete covering of the glove with a single large gel layer. The gel pad 702 is shaped to have finger extensions that extend to and cover a lower portion of the finger areas of the glove 700 so that a user can still firmly grip an object with the medial and distal portions of the users fingers (i.e. distance is defined with respect to the user's palm) while the proximal portion of the glove fingers have padding to reduce vibrations. In this example, there are 4 extensions for the gel pad 702. The gel pads 704 and 706 can be shaped to accommodate the lower portions of a user's palms. In an alternative embodiment, one or more gel pads may be placed at the finger regions of the glove 700. In an alternative embodiment, one or more gel pads may be disposed at the cuff region of the glove. Alternatively, there may be a single gel pad that encircles the cuff region of the glove or covers the cuff region that is on the same side as the palm region of the glove.

Referring now to FIG. 7B, shown therein is glove 720 which comprises an outer surface 721 having separate finger gel pads 722a, 722b, 722c and 722d, separate upper palm gel pads 734a, 734b, 734c, 734d and lower pal gel pads 736a and 736b. There is no complete covering of the glove with a single large gel layer. While only finger gel pad 722a is further labelled for illustrative purposes, finger gel pad 722a comprises gel pads 724, 728 and 732 for the distal, intermediate (i.e. medial) and proximal phalangeal (i.e. finger) portions of the glove 720, respectively, connected to one another with interconnecting gel pads 726 and 728. For example, interconnecting gel pad 726 connects the distal and intermediate finger gel pads 724 and 728 and interconnecting gel pad 730 connects the intermediate and proximal finger gel pads 728 and 732. The lower palm gel pads 736a and 736b have different shapes than those used for glove 700.

Referring now to FIG. 7C, shown therein is glove 750 which comprises a gel substrate/layer 751 that covers an entire surface of the user's hand when they wear the glove 750. The gel substrate 751 includes finger gel pads 722a, 722b, 722c and 722d, upper palm gel pads 734a, 734b, 734c and 734d and lower gel pads 736a and 736b. In this example, the elements of the finger gel pads 722a, 722b, 722c, and 722d are not connected together. This helps to improve manual dexterity and allow for better motion of the hand inside the glove. For example, the finger gel pad 722a only comprises distal, intermediate and proximal finger gel pads 724, 728 and 732, respectively. Also the components of the finger gel pads 722a, 722b, 722c, and 722d are larger than those used for glove 720. The upper palm gel pads 734a, 734b, 734c and 734d are also oriented in a different direction compared to those used for glove 720. Also, the lower gel pads 736a and 736b have a different size and shape compared to those used for gloves 720 and 700. The various gel pads of the glove 720 can be made from the same gel material as the gel substrate layer 751 and be regions where the gel material was molded to be thicker. Alternatively, in some embodiments, the various gel pads of the glove 720 can be made using a different gel composition that has different gel properties based on the location of a particular gel pad on the glove 750 or the particular application for which the glove 750 is used. The different gel properties may help reduce vibrations in various frequency spectrums.

Referring now to FIG. 7D, shown therein is a glove insert 770 which has a gel substrate layer 771 with an outer surface 771. The gel substrate 771 covers the entire surface of the user's hand when they wear a glove having the glove insert 770. The glove insert 770 has a single upper palm gel pad 774 and two lower palm gel pads 776 and 778 that are disposed on the gel substrate layer 771. The flatter regions of the gel substrate 771 can be used to provide a first amount of vibration dampening while the pads 774, 776 and 778 can be used to provide an additional amount of vibration dampening. As with the glove 750, the gel pads 774, 776 and 778 can be made from the same gel material as the gel substrate layer 771 and can be regions where the gel material was molded to be thicker. Alternatively, the gel pads 774, 776 and 778 can be made using a different gel composition that has different gel properties based on the location of a particular gel pad on the glove insert 770 or the particular application for which the glove insert 770 is used. The different gel properties may help reduce vibrations in various frequency spectrums which allows the glove insert 770 to better dampen an array of frequency vibrations across the spectrum that may be expected when the glove insert is used in a glove for a given tool or application. The gel pads 774, 776 and 778 are shaped differently from the gel pads 702, 704 and 706 since the glove insert 770 may be used in different applications than the glove 700. The glove insert 770 may be attached to a glove material inside the glove so that it is covered by other glove material or it may form the lower outer surface of the glove (i.e. the term lower is meant to convey the palm surface of the glove).

It should also be noted that for the glove 750 and the glove inset 770, there can be embodiments in which there are additional layers of the gel substrate or the gel pads sized to be within the ISO standards for maximum allowable thickness and used to provide additional vibrational dampening.

Also, in some of the glove embodiments described herein, there can be alternative embodiments in sensors and appropriate electronic components and circuitry potentially including wiring and contacts can be embedded into some portion of the gel substrate or gel pads used in the gloves depending on which of gloves 700, 720, 750 and 770 are modified to include embedded sensors. Gloves with at least one embedded sensor can be used to give the user estimates of risk and exposure to vibrations.

### Example 3: Polymer Gel Products with Embedded Sensors

For polymer gel products with embedded sensors and/or other electrical components, there are various parameters that may be considered during manufacture such as: (1) encapsulating sensors in the VEP gel (e.g. pouring using different gel formulas to ensure certain comfort levels for the user, certain benefits, and certain product features); (2) functionality and type of sensors (e.g. the gel mold is fitted with various support structures/materials to hold the selected sensors in certain places as the liquid gel is introduced into the gel mold so that the sensors function when encapsulated in gel, and selecting and locating the support structures/materials in the gel molds so that the thickness of the gel above and below the sensor(s) do not affect their function (in other words, the embedded sensor(s) will function as intended after the gel product is produced); and (3) selecting appropriate processing heat levels (e.g. temperature is monitored while embedding the sensors in the liquid VEP gel to ensure that sensors function properly when in use).

The amount of (i.e. thickness of) the gel material above and below a given sensor is integral to the devices' design. Two different liquid gel pour methods/procedures may be used to embed the sensor(s) and the method that is used depends on the type of sensor to be embedded. At least one of these procedures can be to produce the other gel-based products that are described herein having at least one embedded sensor.

The first liquid gel pour method is a one-pour process. With this process, each sensor to be embedded in the gel material is appropriately placed/located in the gel mold prior to pouring the liquid gel and is supported in place by a removal structure/support. Once the hot gel has been poured to the intended thickness/depth and the gel has set or nearly set the sensor support structure is removed. The now embedded sensor will forever maintain/keep its' place in the gel-based device once the gel ‘sets’. The support structures can be made of a material that easily releases from the gel either before or after the gel sets. Such materials may be steel or aluminum formed as a pin such that it supports but is not permanently attached to the sensor.

The second liquid gel pour method is a two-pour process. With this process, each sensor to be embedded in the gel material is attached to a flexible piece of material and the piece of material with the attached sensor(s) will be laid on the top surface of a set or nearly set layer of poured gel. Once the material with the sensor(s) attached is in place at the intended depth, a new layer of gel will be poured over the material with the sensor(s) such that the new layer of gel has a selected thickness/depth and covers/embeds the sensor(s). A material such as Fabric accord 2 oz Non-woven polypropylene olefin can be used as it provides the desired flexibility and will not affect the desired performance of the embedded sensors. Once the second poured gel layer has set, the sensors attached to the material will be permanently located in their intended place/location within the gel-based device.

It should be noted that the techniques for embedding one or more sensors in the gel material described in accordance with the teachings herein are applicable to other types of electronic components/circuitry that are to be embedded in a gel product.

### Example 4: Polymer Gel Pressure Pad

For a polymer gel pressure pad, there are various parameters that may be considered during manufacture including: (1) selecting a gel formulation to achieve certain features for the pad, which may include making two separate liquid gels with different properties and double pouring into a mold; 2) mold design and configuration (e.g. width of chevrons, depth of valleys, drainage ports, and reinforced backing material and (3) testing parameters (e.g. for testing for sheer, friction, and impact properties of the VEP gel pad). The chevrons may be right-side up or upside down V shaped channels that have a certain depth (i.e. referred to as “depth of valleys”) and are distributed along the support pad to allow the weight of the portion of the user's body making contact with the support pad to be better distributed to provide better support to the user, to more effectively distribute pressure and to reduce the occurrence of pressure ulcers. In at least some embodiments, a portion of the chevrons and/or other channels that are used can lead to a drainage port disposed at various areas of the support pad including the edge of the support pad to allow for drainage of any liquids, such as sweat, from the user's body part that is being supported by or is in contact with the gel pad. There may be other surface designs such as using straight lined channels arranged as columns or rows.

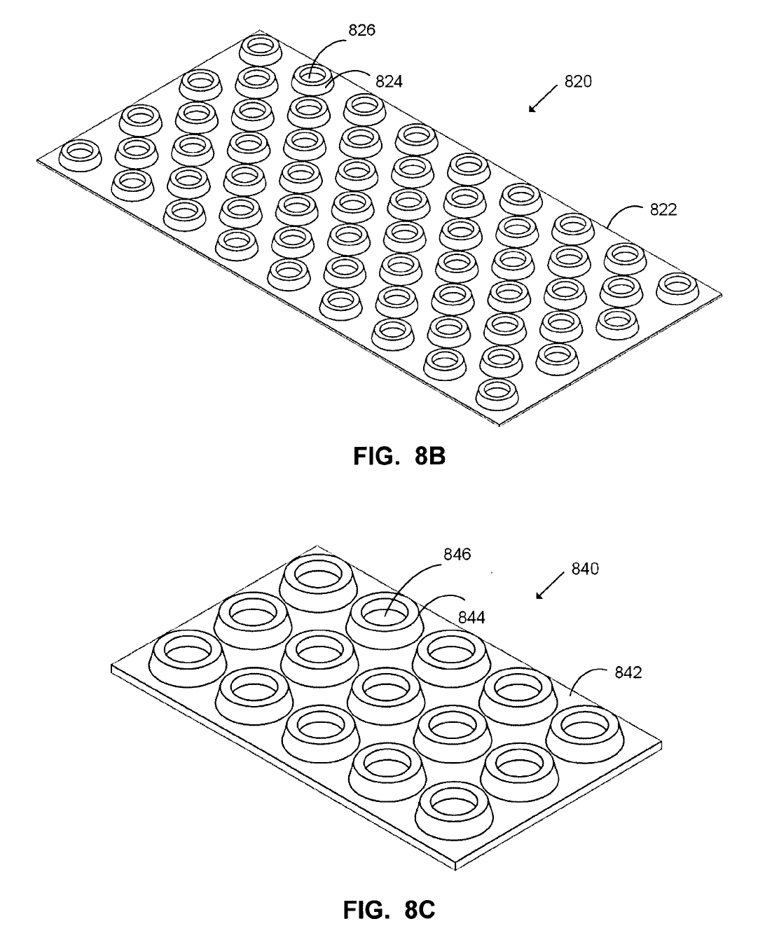

For example, FIG. 8A shows an image of an example embodiment of a gel support pad 800 with a gel substrate layer 802 and an example pattern of chevrons 804 and linear channels 806 that may be used. Chevrons may be used on their own, or linear channels may be used on their own, or a combination of chevrons and linear channels may be used. For example, a pattern of linear channels may be on either side of a pattern of chevrons or two patterns of chevrons may be on either side of a pattern of linear channels (as shown in FIG. 8A) or an alternating pattern of chevrons and linear channels may be used. The linear channels may extend vertically or horizontally and may run along a substantial vertical or horizontal length of the gel pad. These patterns of channels may be used for the surface of other gel products described in accordance with the teachings herein that make contact with the user, such as impact pads, mattresses, and mattress toppers, for example.

In some embodiments, other shape patterns may be used. For example, referring now to FIGS. 8B-8C, shown therein are perspective views of two example embodiments of support pads 820 and 840 with gel substrate layers 802 and 842, respectively, with patterns of circular ridges. For the gel support pad 820, there are diagonal lines of circular ridges 824 with each circular ridge 824 having an interior channel or aperture 826. For the gel support pad 840, there is an alternative pattern of circular ridges 844 with each circular ridge 844 being arranged in a rectilinear array format and each circular ridge 844 having an interior channel or aperture 846. In an alternative embodiment, at least some or all of the circular ridges 824 can be solid so there is no aperture 826. Likewise, in an alternative embodiment, at least some or all of the circular ridges 844 can be solid so there is no aperture 846 and in this case the circular ridges can be referred to as solid circular ridges.

It should be understood that in alternative embodiments, the gel pads 800, 820 and 840 can have at least one embedded sensor and associated electronic circuitry in accordance with the teachings herein.

### Example 5: Gel Surface Finishes for Various Polymer Gel Products

For example, for a given VEP gel product with a certain surface finish, various factors may be considered including: 1) for a natural un-treated surface finish: various degrees of adhesiveness depending on the gel formulation, which allows for wicking of liquid from the VEP gel material if the VEP gel material and user's skin come into direct contact during use, and a soothing feeling for the user; 2) a powdered surface finish lessens the amount of adhesion and reduces wicking, the powder can be reapplied if/when the powdered surface is washed; and the powder can be scented, which may reduce wicking; 3) using a textile/material covering over the entirety of the VEP gel material or placed adjacent to the VEP gel material involves selecting an amount of stretch or rigidity to control/minimize/permit material wicking, when the liquid VEP gel is hot poured directly on the material, the material may protect one surface of the VEP gel material and a second finish can be applied to the other surface of the VEP gel material that is not covered by the textile/material; and 4) applying a sealant to one or more surfaces of the VEP gel material which involves brushing or spraying the sealant on one or more surfaces of the VEP gel material and allowing it to dry; choosing a sealant based on the fact that a particular sealant may reduce the functional elasticity of the gel as the sealant doesn't have the comparable elasticity of a gel, selecting a particular sealant for downward pressure reduction (e.g. pressure placed vertically to the surface); modifying the sealant to allow for ease of application by diluting to a desired consistency and selecting a material that can then be placed on top of the dried sealant (any suitable material can be selected).

While the applicant's teachings described herein are in conjunction with various embodiments for illustrative purposes, it is not intended that the applicant's teachings be limited to such embodiments as these the embodiments described herein are intended to be examples. On the contrary, the applicant's teachings described and illustrated herein encompass various alternatives, modifications, and equivalents, without departing from the embodiments described herein, the general scope of which is defined in the appended claims.

1.-25. (canceled)

26. A method of embedding at least one sensor into a viscoelastic polymer gel substrate, the method comprises:
placing a support structure for the at least one sensor into a mold;
placing the at least one sensor on the support structure;
heating and processing a slurry comprising a mixture of polymer and oil to a desired temperature to produce a liquid viscoelastic gel liquid;
inserting the gel liquid into the mold; and
allowing the gel liquid to set to form a solid viscoelastic polymer gel having at least one embedded sensor.

27. The method of claim 26, wherein the method further comprises adding one or more additives to the slurry for modifying one or more physical characteristics of the solid viscoelastic polymer gel.

28. The method of claim 26, wherein the method further comprises adding additional electronics to the at least one sensor in the mold and establishing electrical connections prior to dispensing the liquid gel material into the mold.

29. The method of claim 26, wherein the method further comprises texturing a receiving surface of the mold to produce a textured surface on the solid viscoelastic polymer gel.

30. The method of claim 29, wherein the texturing comprises forming at least one chevron pattern, at least one linear channel pattern, a pattern of circular ridges, a pattern of solid circular ridges, or a combination pattern having chevrons and linear channels on the textured surface.

31. The method of claim 29, wherein the method comprises forming a plurality of air pockets on the textured surface.

32. The method of claim 26, wherein after the liquid gel has been poured into the mold up to a selected thickness and the liquid gel is nearly set the support structure is removed.

33. The method of claim 32, wherein the support structure is made from steel or aluminum formed as a pin such that it supports but is not permanently attached to the at least one sensor.

34. The method of claim 26, prior to placing the support structure into the mold a first layer of gel liquid is poured, the at least one sensor is attached to a flexible piece of material and placed on the first layer of gel when the first layer of gel sets or nearly sets, and a new layer of gel is poured over the flexible piece of material such that the new layer of gel has a selected thickness and covers the at least one sensor.

35. The method of claim 34, wherein the flexible piece of material comprises fabric or another flexible material.

36. The method of claim 26, wherein after the gel is set, the method comprises injecting a foam mixture which rises and expands to fill the mold to form a gel injected foam product and/or the method comprises using a contoured mold to provide a contour to the gel and foam portions of the gel injected foam product.

37. (canceled)

38. A compression wrap comprising:
a gel band that is stretchable and has first and second surfaces and first and second end portions; and
a backing member adjacent to one of the first and second surfaces of the gel band, wherein the backing member is stretchable in a similar manner as the gel band.

39. The compression wrap of claim 38, wherein the gel band comprises viscoelastic polymers and/or the gel band further comprises one or more additives for modifying one or more physical characteristics of the gel band.

40. (canceled)

41. (canceled)

42. The compression wrap of claim 38, wherein the backing member is removable from the gel band for use and is re-attachable after use for storage.

43. The compression wrap of claim 38, wherein the gel band is self-adhering.

44. The compression wrap of claim 38, wherein the backing member comprises at least one of a fabric material including one or more of polyester, rayon, spandex, and nylon.

45. The compression wrap of claim 38, wherein the gel band has an elasticity to enable deformation in the range of 2:1 to 10:1 to provide a sufficient compressive force when the compression wrap is wrapped around an anatomical location of a user.

46. The compression wrap of claim 45, wherein the gel band has a thermal conductivity in the range of 4.0 to 6.0 Watts per meter Kelvin (W/(m·K)) to allow the gel band to maintain a desired temperature over a desired period of time when the compression wrap is chilled or heated prior to being wrapped around the anatomical location of the user.

47. The compression wrap of claim 38, further comprising at least one sensor embedded within the gel band, the at least one sensor comprising at least one of a pressure sensor, a force sensor, a temperature sensor, an accelerometer and a light sensor.

48. (canceled)

49. The compression wrap of claim 47, wherein the at least one sensor is embedded proximally to the second surface of the gel band, the second surface being placed adjacent to a surface of a user during use to facilitate measurements from the user, and/or the at least one sensor is embedded near a first end of the band, wherein the first end is applied to the body part first.

50. The compression wrap of claim 47, wherein the at least one sensor comprises an interface module, and during use, the at least one sensor senses information and the interface module communicates the sensed information to a receiver module via wired or wireless communication.

51.-67. (canceled)
